(19) United States
(12) Patent Application Publication
(10) Pub. No.: US 2024/0317816 A1
(43) Pub. Date: Sep. 26, 2024

(54) PEPTIDES FOR IMMUNOTHERAPY

(71) Applicant: Genevive, Inc., San Mateo, CA (US)

(72) Inventors: Helena Kiefel, San Mateo, CA (US); Dhwani Haria, San Mateo, CA (US); Michi Izumi Willcoxon, San Mateo, CA (US); Kyle Roskamp, San Mateo, CA (US); Toshihiko Takeuchi, San Mateo, CA (US); Karim Dabbagh, San Mateo, CA (US)

(21) Appl. No.: 18/703,043

(22) PCT Filed: Jun. 3, 2022
(86) PCT No.: PCT/US2022/032236

§ 371 Date: Apr. 19, 2024

Related U.S. Application Data
(60) Provisional application No. 63/286,741, filed on Dec. 7, 2021.

(30) Foreign Application Priority Data
Jun. 4, 2021 (WO) PCT/US2021/035983

(51) Int. Cl. C07K 14/195; A61K 38/00; A61K 39/00; A61P 35/00; C07K 16/28

(52) U.S. Cl. CPC C07K 14/195; A61P 35/00; C07K 16/2818; A61K 38/00; A61K 2039/505; C07K 2319/02

(57) ABSTRACT

The disclosure provides peptides, pharmaceutical compositions, and methods of producing thereof. Such peptides can be useful, for example, in treating various human diseases such as immunological diseases or cancers. In some embodiments, the peptides are useful as immunotherapeutics for modulating regulatory and effector molecules of the mammalian immune system.

### CLAIM OF PRIORITY

This application claims priority to P.C.T. Patent Application PCT/US2021/035983, filed on Jun. 4, 2021, and to U.S. Provisional Application Ser. No. 63/286,741, filed on Dec. 7, 2021, which is incorporated by reference in its entirety herein.

### SEQUENCE LISTING

This application contains a Sequence Listing that has been submitted electronically as an ASCII text file named 47192-0062WO1-ST25.txt. The ASCII text file, created on Jun. 3, 2022, is 28.3 kilobytes in size. The material in the ASCII text file is hereby incorporated by reference in its entirety.

### TECHNICAL FIELD

The present disclosure is related to peptides and compositions thereof, and using such peptides and compositions thereof for treating a disease in a subject associated with the adaptive and innate immune system and immunology-associated disorders.

### BACKGROUND

Inflammatory and immune-related diseases are the manifestation or consequence of complex and often multiple interconnected biological pathways, which in normal physiology are critical to respond to injury or insult, initiate repair from injury or insult, and mount an innate and/or acquired defense against foreign organisms. Disease or pathology can occur when these normal physiological pathways cause additional injury or insult that can be directly related to the intensity of the response, as a consequence of abnormal regulation or excessive stimulation, as a reaction to self, or as a combination thereof.

While the genesis of these diseases often involves multistep pathways and often multiple biological systems or pathways, intervention at critical points in one or more of these pathways can have an ameliorative or therapeutic effect. Therapeutic intervention can occur by either antagonism of a detrimental process/pathway or stimulation of a beneficial process/pathway. Many immune-related diseases are known and have been extensively studied. Such diseases include inflammatory diseases, infectious diseases, immunodeficiency diseases, neoplastic diseases (e.g., cancer), etc.

Cancer is the second leading cause of death, resulting in one out of every four deaths in the United States. More than one million people in the Unites States are diagnosed with cancer each year, and in 2016, it was estimated that 595,690 cancer deaths occurred. Due to the ever-increasing aging population in the United States, it is reasonable to expect that rates of cancer incidence will continue to grow. See, e.g., American Cancer Society.

Cancer involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer.

Cancer is currently treated using a variety of modalities including surgery, radiation therapy and chemotherapy. The choice of treatment will depend upon the type, location and dissemination of the cancer. For example, surgery and radiation therapy may be used to treat non-solid tumor cancers such as leukemia and lymphoma. One of the advantages of surgery and radiation therapy is the ability to control to some extent the impact of the therapy, and thus to limit the toxicity to normal tissues in the body. However, surgery and radiation therapy are often followed by chemotherapy to guard against any remaining or radio-resistant cancer cells. Chemotherapy is also the most appropriate treatment for disseminated cancers such as leukemia and lymphoma, as well as metastases.

Because many chemotherapy agents target cancer cells based on their proliferative profiles, tissues such as the gastrointestinal tract and the bone marrow which are normally proliferative are also susceptible to the effects of the chemotherapy.

Many chemotherapeutic agents have been developed for the treatment of cancer. Not all tumors, however, respond to chemotherapeutic agents and others, although initially responsive to chemotherapeutic agents, may develop resistance. As a result, the search for effective anti-cancer drugs has intensified in an effort to find even more effective agents with less non-specific toxicity.

Thus, there is a great need to develop additional and safer cancer therapeutics that leverage aspects of the mammalian immune system to aid in treating and preventing cancer. Despite new cancer treatments coming to market each year, these treatments are further accompanied by problematic side effects.

### SUMMARY

This disclosure is based, at least in part, on the identification of peptides, nucleic acid constructs, and pharmaceutical compositions that are immunotherapies, and methods of treating a disease (e.g., cancer) using the peptides, nucleic acids, and pharmaceutical compositions disclosed herein.

Provided herein are peptides that may include or consist of an amino acid sequence described herein, e.g., in a table set forth herein, e.g., an amino acid sequence having at least 85%, 90%, 95%, 97%, 98%, 99%, or 100% sequence identity to any one of SEQ ID NOs: 1-65, preferably SEQ ID NO:46 (GQRQKHKPNECIVMVRGKRRIIKCKECMPKVSPRVKM). Also provided herein are peptides that may include or consist of an amino acid sequence described herein, e.g., in a table set forth herein, e.g., an amino acid sequence having at least 85%, 90%, 95%, 97%, 98%, 99%, or 100% sequence identity to SEQ ID NO: 51 (LGKTDWIEKYFKVKKEKIDKMQRFLQG). For example, the peptide can have an amino acid sequence having at least 90%, at least 95%, or at least 99% sequence identity to an amino acid sequence described herein, e.g., in a table set forth herein, e.g., any one of SEQ ID NOs: 1-65, preferably SEQ ID NO:46 (GQRQKHKPNECIVMVRGKRRIIKCKECMPKVSPRVKM) or SEQ ID NO: 51 (LGKTDWIEKYFKVKKEKIDKMQRFLQG). In some cases, the peptide has an amino acid sequence described herein, e.g., in a table set forth herein, e.g., the amino acid sequence of any one of SEQ ID NOs: 1-65, preferably SEQ ID NO:46 (GQRQKHKPNECIVMVRGKRRIIKCKECM PKVSPRVKM). In some cases, the peptide has an amino acid sequence described herein, e.g., in a table set forth herein, e.g., the amino acid sequence of SEQ ID NO: 51 (LGKTDWIEKYFKVKKEKIDKMQRFLQG). In some cases, the peptide contains at least one D-amino acid. In some cases, the peptide consists of D-amino acids.

Also provided herein are nucleic acid constructs including a polynucleotide, wherein the polynucleotide encodes any of the peptides disclosed herein.

Also provided herein are recombinant host cells that may have an exogenous polynucleotide, the polynucleotide encoding peptide having at least 85%, 90%, 95%, 97%, 98%, 99%, or 100% sequence identity to any one of SEQ ID Nos: 1-65. In some cases, the exogenous polynucleotide further encodes a host cell specific signal sequence. In some cases, the exogenous polynucleotide further encodes a heterologous promoter. In some cases, the heterologous promoter is a constitutive promoter. In some cases, the heterologous promoter is an inducible promoter. In some cases, the recombinant host cell is a prokaryotic cell, a eukaryotic cell, or a fungal cell. In some cases, the recombinant host cell is selected from the group consisting of: an Escherichia coli cell, a Lactococcus lactis cell, a Streptomyces coelicolor cell, a Streptomyces lividans cell, a Streptomyces albus cell, a Streptomyces venezuelae cell, or a Bacillus subtilis cell. In some cases, the recombinant host cell is a Saccharomyces cerevisiae cell, a Pichia pastoris cell, a Yarrowia lipolytica cell, an Aspergillus niger cell, or a Hansenula polymorpha cell. In some cases, the eukaryotic cell is a Chinese Hamster Ovary cell.

Also provided herein are pharmaceutical compositions that may include a) a peptide having at least 85%, 90%, 95%, 97%, 98%, 99%, or 100% sequence identity to any one of SEQ ID NOs: 1-65, any of the nucleic acid constructs described herein, or a plurality of any of the recombinant host cells described herein; and b) a pharmaceutically acceptable carrier. In some cases, the pharmaceutical composition is formulated for oral administration, rectal administration, intravenous administration, or intratumoral administration. In some cases, the pharmaceutical composition is formulated as a tablet, a capsule, a powder, or a liquid. In some cases, the composition is formulated as a tablet. In some cases, the tablet is coated. In some cases, the coating comprises an enteric coating.

Also provided herein are methods of modulating the activity of one or more target proteins in a subject, the method comprising administering to the subject: a) a peptide having at least 85%, 90%, 95%, 97%, 98%, 99%, or 100% sequence identity to any one of SEQ ID NOs: 1-65, and/or b) any peptide disclosed herein, and/or c) any nucleic acid construct disclosed herein, and/or d) a plurality of any recombinant host cells disclosed herein, and/or e) any pharmaceutical composition disclosed herein; wherein the one or more target protein is selected from the group consisting of a CCR9 protein, a CXCR3 protein, and a CXCR4 protein. In some cases, the CCR9 protein and/or the CXCR3 protein has an activity that is increased. In some cases, the CXCR4 protein has an activity that is decreased. In some cases, the activity is measured with a beta-arrestin reporter cell assay as described herein.

Also provided herein are methods for treating a disease in a subject in need thereof, the method comprising administering to the subject: a) a peptide having at least 85%, 90%, 95%, 97%, 98%, 99%, or 100% sequence identity to any one of SEQ ID NOs: 1-65, and/or or b) any peptide disclosed herein, and/or c) any nucleic acid construct disclosed herein, and/or d) a plurality of any recombinant host cells disclosed herein, and/or e) any pharmaceutical composition disclosed herein.

In some cases, the disease is a neoplasm. In some cases, the disease is cancer. In some cases, the cancer is at least one selected from the group consisting of: melanoma, renal cell carcinoma, non-small cell lung carcinoma, basal cell carcinoma, biliary tract cancer, bladder cancer, bone cancer, brain and central nervous system cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, small-cell lung cancer, Hodgkin's lymphoma, non-Hodgkins lymphoma, myeloma, neuroblastoma, oral cavity cancer, ovarian cancer, pancreatic cancer, prostate cancer, retinoblastoma, rhabdomyosarcoma, rectal cancer, cancer of the respiratory system, sarcoma, skin cancer, stomach cancer, testicular cancer, thyroid cancer, uterine cancer, and cancer of the urinary system.

Also provided herein are methods for increasing the response to an immunotherapy in a subject in need thereof comprising administering to the subject a composition, wherein the composition comprises: a) a peptide having at least 85%, 90%, 95%, 97%, 98%, 99%, or 100% sequence identity to any one of SEQ ID NOs: 1-65, and/or or b) any peptide disclosed herein, and/or c) any nucleic acid construct disclosed herein, and/or d) a plurality of any recombinant host cells disclosed herein, and/or e) any pharmaceutical composition disclosed herein.

In some cases, the peptide modulates the production of at least one cytokine in the subject. In some cases, the cytokine is selected from the group consisting of TNF-α, IL-17, IL-1β, IL-2, IFN-γ, IL-6, IL-12, IL-25, IL-33, IL-8, MCP-1, MIP-3α, CXCL1, IL-23, IL-4, IL-10, IL-13, IFN-α, and TGF-β. In some cases, the peptide induces the production of at least one pro-inflammatory cytokine in the subject. In some cases, the at least one pro-inflammatory cytokine is selected from the group consisting of TNF-α, IL-17, IL-1β, IL-2, IFN-γ, IL-6, IL-12, IL-25, IL-33, IL-8, MCP-1, MIP-3α, CXCL1, and IL-23.

In some cases, the peptide suppresses the production of at least one anti-inflammatory cytokine in the subject. In some cases, the at least one anti-inflammatory cytokine is selected from the group consisting of IL-4, IL-10, IL-13, IFN-α, and TGF-β. In some cases, the peptide increases Th1 activation in the subject. In some cases, the peptide increases dendritic cell maturation in the subject. In some cases, the peptide increases CD70 expression in the subject. In some cases, the peptide increases the clonal expansion of T_{eff} in the subject.

In some cases, the peptide increases activity of a CCR9 protein or a CXCR3 protein. In some cases, the peptide decreases activity of a CXCR4 protein. In some cases, the peptide binds to a CCR9 protein, a CXCR3 protein, or a CXCR4 protein. In some cases, any of the methods described herein further comprise administering an additional treatment for cancer, and/or other adjunct therapy to the subject.

Also provided herein are methods of treating cancer in a subject in need thereof, the method comprising a) administering to the subject: i) a peptide having at least 85%, 90%, 95%, 97%, 98%, 99%, or 100% sequence identity to any one of SEQ ID NOs: 1-65, and/or or ii) any peptide disclosed herein, and/or iii) any nucleic acid construct disclosed herein, and/or iv) a plurality of any recombinant host cells disclosed herein, and/or v) any pharmaceutical composition disclosed herein; and b) administering to the subject an additional treatment for cancer, and/or adjunct therapy to the subject.

In some cases, the additional treatment for cancer and/or adjunct therapy comprises a probiotic. In some cases, the additional treatment for cancer and/or adjunct therapy comprises surgery, radiation therapy, or a combination thereof. In some cases, the additional treatment for cancer and/or adjunct therapy comprises a therapeutic agent.

In some cases, the therapeutic agent comprises a chemotherapeutic agent, a targeted therapy, an additional immunotherapy, or a combination thereof. In some cases, the chemotherapeutic agent comprises carboplatin, cisplatin, gemcitabine, methotrexate, paclitaxel, pemetrexed, lomustine, temozolomide, dacarbazine, or a combination thereof.

In some cases, the targeted therapy comprises afatinib dimaleate, bevacizumab, cetuximab, crizotinib, erlotinib, gefitinib, sorafenib, sunitinib, pazopanib, everolimus, dabrafenib, aldesleukin, interferon alfa-2b, ipilimumab, peginterferon alfa-2b, trametinib, vemurafenib, or a combination thereof.

In some cases, the additional immunotherapy comprises a cell therapy, a therapy with an immune checkpoint inhibitor, a therapy with a co-stimulatory immune checkpoint agent, or a combination thereof. In some cases, the immune checkpoint inhibitor is selected from the group consisting of: ipilimumab, nivolumab, pembrolizumab, atezolizumab, avelumab, durvalumab, cemiplimab, and a combination thereof. In some cases, the co-stimulatory immune checkpoint agent is selected from the group consisting of IBI101, utomilumab, MEDI1873, and a combination thereof. In some cases, the cell therapy is a CAR T cell therapy.

In any of the methods described herein, the subject can be a human.

Also described herein are methods of producing a peptide including culturing any of the recombinant host cells described herein, under conditions sufficient for expression of the encoded peptide.

Also described herein are methods of producing a peptide, the method comprising chemically synthesizing the peptide of any one of SEQ ID NOs: 1-65.

Also provided herein are peptides comprising the amino acid sequence set forth in, X₁KX₃X₄X₅SVKX₉X₁₀CX₁₂X₁₃CX₁₄X₁₅X₁₆IX₁₈RX₂₀GX₂₂X₂₃X₂₄X₂₅IX₂₇X₂₈X₂₉PX₃₁HKQX₃₅QX₃₇ (SEQ ID NO:45), wherein X₁ is optional, each of X₂-X₂₅ and X₂₈-X₃₅ is independently a naturally occurring amino acid, X₂₇ is selected from the group consisting of C and CP; and X₃₇ is selected from the group consisting of G, GN, and DRH.

In peptides having the sequence set forth in SEQ ID NO: 45 X₁ is the amino acid M, and/or X₃ is an amino acid selected from the group consisting of V, I, and T, and/or X₄ is an amino acid selected from the group consisting of: R, K, and Q, and/or X₅ is an amino acid selected from the group consisting of: P, S, and A, and/or X₉ is an amino acid selected from the group consisting of P, T, and K, and/or X₁₀ is an amino acid selected from the group consisting of: M and I, and/or X₁₂ is an amino acid selected from the group consisting of: E and D, and/or X₁₃ is an amino acid selected from the group consisting of: K and Y, and/o X₁₅ is an amino acid selected from the group consisting of: K and R, and/or X₁₆ is an amino acid selected from the group consisting of: V and I, and/or X₁₈ is an amino acid selected from the group consisting of: K and R, and/or X₂₀ is an amino acid selected from the group consisting of: K, N, and H, and/or X₂₂ is an amino acid selected from the group consisting of: R, K, H, S, and I, and/or X₂₃ is an amino acid selected from the group consisting of: V and I, and/or X₂₄ is an amino acid selected from the group consisting of: M, R, A, and L, and/or X₂₅ is an amino acid selected from the group consisting of: V and I, and/or X₂₈ is selected from the group consisting of: E, Q, A, and T, and/or X₂₉ is an amino acid selected from the group consisting of: N and E, and/or X₃₁ is an amino acid selected from the group consisting of K and R, and/or X₃₅ is an amino acid selected from the group consisting of: K and R.

A peptide having a sequence of SEQ ID NO: 45, or any of the variants thereof described herein, can modulates activity of a CCR9 protein, a CXCR3 protein, or a CXCR4 protein.

A peptide having a sequence of SEQ ID NO: 45, or any of the variants thereof described herein, binds to a CCR9 protein, a CXCR3 protein, or a CXCR4 protein.

The term “subject” refers to a mammal such as a human, a non-human primate, a livestock animal (e.g., bovine, porcine), a companion animal (e.g., canine, feline) and a rodent (e.g., a mouse and a rat). In some embodiments, the term refers to a human subject.

As used herein, unless otherwise noted, the terms “treating,” “treatment,” and the like, shall include the management and care of a subject (e.g., a mammal such as a human) for the purpose of combating a disease, condition, or disorder and includes the administration of a disclosed peptide to alleviate the symptoms or complications, or reduce the rate of progression of the disease, condition, or disorder. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, and/or condition.

The term “T cell-mediated disease” means a disease in which T cells directly or indirectly mediate. The T cell-mediated disease may be associated with, but not limited to, cell-mediated effects, lymphokine-mediated effects, and/or effects associated with B cells if the B cells are stimulated, for example, by the lymphokines secreted by T cells.

A “peptide” as used herein refers to a polypeptide having 3 to 50 amino acids. For example, a peptide can have 10 to 50 amino acids, 20 to 40 amino acids, 20 to 30 amino acids, 25 to 35 amino acids, or 30 to 40 amino acids. In some embodiments, a peptide can be produced recombinantly. In some embodiments, a peptide can be produced by chemical synthesis.

The term “pharmaceutically acceptable salt,” as used herein, represents salts or zwitterionic forms of the peptides, proteins, or compounds of the present disclosure, which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartic acid, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamic acid, bicarbonate, para-toluenesulfonate, and undecanoate. Also, amino groups in the compounds of the present disclosure can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt can suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R¹)(R²)(R³)(R⁴)⁺, where R¹, R², R³ and R⁴ independently will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases can also be formed, e.g., hemisulphate and hemicalcium salts.

As used herein, the term “therapeutically effective amount” refers to an amount of a therapeutic agent (e.g., a peptide, polypeptide, or protein of the disclosure), which confers a therapeutic effect on the treated subject. Such a therapeutic effect can be objective (i.e., measurable by some test or marker) or subjective (i.e., subject gives an indication of, or feels an effect). In some embodiments, “therapeutically effective amount” refers to an amount of a therapeutic agent or composition effective to treat or ameliorate a relevant disease or condition, and/or to exhibit a detectable therapeutic or preventative effect, such as by ameliorating symptoms associated with the disease and/or also lessening severity or frequency of symptoms of the disease. For any particular therapeutic agent, a therapeutically effective amount (and/or an appropriate unit dose within an effective dosing regimen) can vary, for example, depending on route of administration or on combination with other therapeutic agents. Alternatively or additionally, a specific therapeutically effective amount (and/or unit dose) for any particular subject can depend upon a variety of factors including the particular form of disease being treated; the severity of the condition or pre-condition; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts. The current disclosure utilizes therapeutically effective amounts of peptides and compositions comprising the same, to treat a variety of diseases, such as cancer

“Pharmaceutical” implies that a composition, reagent, method, and the like, are capable of a pharmaceutical effect, and also that the composition is capable of being administered to a subject safely. “Pharmaceutical effect,” without limitation, can imply that the composition, reagent, or method, is capable of stimulating a desired biochemical, genetic, cellular, physiological, or clinical effect, in at least one subject, such as a mammalian subject, for example, a human, in at least 5% of a population of subjects, in at least 10%, in at least 20%, in at least 30%, in at least 50% of subjects, and the like.

The phrases “pharmaceutical” or “pharmacologically acceptable” or “pharmaceutically acceptable” refer to molecular entities and compositions suitable for administration to a subject, such as, for example, a human, as appropriate. For example, “pharmaceutical” or “pharmacologically acceptable” or “pharmaceutically acceptable” can refer to agents approved by a regulatory agency of the Federal or a state government or listed in the U.S. Pharmacopoeia or other generally recognized pharmacopoeia for safe use in animals, and more particularly safe use in humans.

“Pharmaceutically acceptable vehicle” or “pharmaceutically acceptable carrier” refers to a diluent, adjuvant, excipient or carrier with which a peptide as described herein is administered. For example, a “pharmaceutically acceptable carrier” can include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

“Prophylaxis” means a measure taken for the prevention of a disease or condition or at least one symptom thereof.

“Preventing” or “prevention” refers to a reduction in risk of acquiring a disease or disorder (i.e., causing at least one of the clinical symptoms of the disease not to develop in a subject that can be exposed to or predisposed to the disease but does not yet experience or display symptoms of the disease, or causing the symptom to develop with less severity than in absence of the treatment). “Prevention” or “prophylaxis” can also refer to delaying the onset of the disease or disorder.

“Prophylactically effective amount” means the amount of a compound, i.e., a peptide as described herein, that when administered to a subject for prevention of a disease or condition, is sufficient to effect such prevention of the disease or condition or to prevent development of at least one symptom of the disease or condition or effect development of the symptom at a lower level of severity than in the absence of administration of the compound. The “prophylactically effective amount” can vary depending on the compound, the disease and its severity, and the age, weight, etc., of the subject to be treated.

The term “amino acid” or “any amino acid” refers to any and all amino acids, including naturally occurring amino acids (e.g., alpha-amino acids), unnatural amino acids, and modified amino acids. It includes both D- and L-amino acids. Non-limiting examples of unnatural amino acids include beta-amino acids, homo-amino acids, proline and pyruvic acid derivatives, 3-substituted alanine derivatives, glycine derivatives, ring substituted phenylalanine and tyrosine derivatives, linear core amino acids, and N-methyl amino acids. A modified amino acid can be an amino acid resulting from a reaction at an amino group, carboxy group, side-chain functional group, or from the replacement of any hydrogen by a heteroatom. Amino acids are referred to herein by their full name and/or by their IUPAC one-letter abbreviation.

The recitations “sequence identity,” “percent identity,” “percent homology,” or for example, “comprising a sequence 50% identical to,” as used herein, refer to the extent that sequences are identical on an amino acid-by-amino acid basis, or a nucleotide-by-nucleotide basis, or over a window of comparison. Thus, a “percentage of sequence identity” can be calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes. The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. 0), using a PAM120 weight residue table, a gap length penalty of 12 and a gap penalty of 4.

Related (and variant) peptides encompass “variant” peptides. Variant peptides differ from another (i.e., parental) peptide and/or from one another by a small number of amino acid residues. A variant can include one or more amino acid modifications (e.g., amino acid deletion, insertion, or substitution) as compared to the parental protein/peptide from which it is derived. In some embodiments, the number of different amino acid residues is any of about 1, 2, 3, 4, 5, 10, or 20. In some embodiments, variants differ by about 1 to about 15 amino acids (e.g., 1 to 5, 1 to 10, 5 to 10, 5 to 15, or 10 to 15). In some embodiments, variants can differ in amino acid length by at least 1 up to about 10, 15, or 20 amino acids. Amino acid length differences because of additional of deletions of individual amino acids or multiple amino acids in a single location or in multiple locations throughout the peptide/protein sequence. Alternatively or additionally, variants can have a specified degree of sequence identity with a reference protein/peptide or nucleic acid, e.g., as determined using a sequence alignment tool, such as the previously discussed BLAST, ALIGN, and CLUSTAL. For example, variant proteins/peptides or nucleic acid can have at least about 35%, 40%, 45%, 50%, 55%, 60%, 65%, 70%7, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence. In some embodiments, variant proteins/peptides or nucleic acids are not 100% identical to a reference sequence.

As used herein, the term “amino acid modification” refers to, e.g., an amino acid substitution, deletion, and/or insertion, as is well understood in the art.

The term “bacterium” or “bacterial cell” means any cell from or derived from any bacterium (e.g., a Gram positive bacterium, or a Gram negative bacterium). Non-limiting examples of bacteria are described herein where appropriate. Additional examples of bacteria are known in the art.

The term “recombinant bacterium” means a bacterium that contains a nucleic acid that is not naturally present in the bacterium. For example, the nucleic acid that is not naturally present in the bacterium can encode a recombinant polypeptide (e.g., any of the exemplary recombinant peptides described herein) and/or can encode a selectable marker (e.g., any of the exemplary selectable markers described herein). The nucleic acid that is not naturally present in the cell can, e.g., be integrated into the genome of the bacterium. In other examples, the nucleic acid that is not naturally present in the cell is not integrated into the genome of the bacterium. For example, a nucleic acid that is not naturally present in the bacterium can be present on a vector.

The term “promoter” is a nucleic acid sequence that is operably linked to a nucleic acid sequence encoding a polypeptide (e.g., a recombinant peptide) that can increase the transcription of the nucleic acid sequence encoding the polypeptide (e.g., a peptide described herein). In some aspects, a promoter is constitutive. In other aspects, a promoter is inducible. Non-limiting examples of promoters are described herein. Additional examples of promoters are known in the art.

The term “culturing” refers to growing a population of cells, e.g., microbial cells, under suitable conditions for growth, in a liquid or solid medium.

The term “purifying” means a step performed to isolate a recombinant peptide from one or more other impurities (e.g., bulk impurities) or components present in a fluid containing a recombinant peptide (e.g., liquid culture medium polypeptides or one or more other components (e.g., DNA, RNA, other polypeptides, endotoxins, viruses, etc.) present in or secreted from a mammalian cell).

The terms “isolated”, “purified”, “separated”, and “recovered” as used herein refer to a material (e.g., a polypeptide, nucleic acid, or cell) that is removed from at least one component with which it is naturally associated, for example, at a concentration of at least 90% by weight, or at least 95% by weight, or at least 98% by weight of the sample in which it is contained. For example, these terms can refer to a material which is substantially or essentially free from components which normally accompany it as found in its native state, such as, for example, an intact biological system, or is substantially or essentially free from other proteins in the system from which it is expressed.

As used herein, the term “host cell” refers to a cell or cell line into which a recombinant expression vector (e.g., a nucleic acid construct) can be introduced for expression of the peptide in the host cell. A host cell comprising a recombinant vector can be referred to as a “recombinant host cell.”

As used herein, “inhibiting and suppressing” and like terms should not be construed to require complete inhibition or suppression, although this can be desired in some embodiments.

Thus, as used herein, the terms “increase” or “reduce,” or grammatical equivalents, indicate values that are relative to a reference (e.g., baseline) measurement, such as a measurement taken under comparable conditions (e.g., in the same subject prior to initiation of treatment described herein, or a measurement in a control subject (or multiple control subjects) in the absence of treatment) described herein. In some embodiments, a suitable control is a baseline measurement, such as a measurement in the same subject prior to initiation of the treatment described herein, or a measurement in a control subject (or multiple control subjects) in the absence of the treatment described herein.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 up to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations,

The details of one or more embodiments of the invention are set forth in the accompanying drawings and the description below. Other features, objects, and advantages of the invention will be apparent from the description and drawings, and from the claims. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is a heat map of relative cytokine levels in the supernatants of low dose LPS-stimulated dendritic cells following treatment with various peptides.

FIG. 2 shows plots of CXCL10 concentrations normalized to 30 μM peptide for various peptides.

FIG. 3. shows a volcano plot illustrating putative binding partners of SG-3-00802 enriched over transferrin ligand.

FIGS. 4A-4E are plots of activation or inhibition of chemokine receptors when incubated with peptides.

FIGS. 5A-5E are plots showing the activation or inhibition of chemokine receptors when incubated with various peptides.

FIGS. 6A-6C are plots of activation of CXCR3 measured in a reporter cell line incubated with CXCL9 (FIG. 6A), CXCL10 (FIG. 6B), or CXCL11 (FIG. 6C) in the presence of SG-3-00802.

FIG. 7 is a plot of the activation of CXCR3 measured in a reporter cell line incubated with CXCL11 in the presence of SG-3-00802 and SG-3-006683 peptides.

FIG. 8A is a plot of the % activation of CXCR3 by CXCL11 with SG-3-00802 in a dose-dependent manner. FIG. 8B is a plot of the % activation of CXCR3 by CXCL11 with SG-3-006683 in a dose dependent manner.

FIG. 9A is a predicted structure of SG-3-00802 peptide with a zinc ion.

FIG. 9B is a plot of binding of peptides to human dendritic cells tested by flow cytometry.

FIGS. 10A-10B are plots of cytokine release after treatment with SG-3-00802 alone or in combination with anti-CD40 and anti-PD-L1.

FIGS. 11A-11B are plots of cytokine release after treatment with SG-3-05021 alone or in combination with anti-CD40 and anti-PD-L1.

FIG. 12A is a plot showing average tumor volume after various treatments with SG-3-00802 and anti-PD-1. Lines in FIGS. 12B-12E represent individual tumors used to obtain averages in FIG. 12A.

FIGS. 13A-13B are plots showing mouse survival curves after treatment with SG-3-00802.

FIGS. 14A-C are plots showing tumor volume in mice over time. Mice were treated previously with SG-3-00802 and/or anti-PD-1, as indicated, and showed tumor regression. Mice were re-injected with tumors and the second tumor size was followed. Tumor sizes of mice previously treated with PBS (FIG. 14A), SG-3-00802 (FIG. 14B), and SG-3-00802+anti-PD-1 (FIG. 14C) are shown.

FIG. 15 is a plot of average tumor volume in mice over time when treated with SG-3-05021 and anti-PD-1.

FIG. 16 is a plot of a survival curve of mice after various treatments with SG-3-05021.

FIGS. 17A-17C are plots of the percent of cells migrating when exposed to various treatments, including peptide SG-3-00802 (PEP802) at indicated concentrations.

FIG. 18 is a plot of the percent of migrating cells as a percentage of the input of cells when exposed to various treatments, including peptide SG-3-006683 at indicated concentrations.

FIGS. 19A-19B are plots of CXCL10 levels in mouse serum (FIG. 19A) and mouse tumors (FIG. 19B). FIG. 19C is a plot of CD8 levels in mouse tumors.

FIG. 20 is a plot of CXCL10 released during treatment with various peptides.

### DETAILED DESCRIPTION

This document provides compositions and methods for treating subjects in need thereof (e.g., subjects having an immunological disease or cancer) using one or more peptides described herein. Immunological diseases and cancers that can be treated using a peptide as described herein can include diseases that are associated with inflammatory immune responses. In some embodiments, peptides described herein modulate immunoregulatory cells, such as increase anti-tumor responses or decrease pro-tumor responses, including but not limited to increasing population sizes of T cells, effector T cells and/or dendritic cells.

### Peptides

Polypeptides encoded by bacterial genomes, such as the Oscillibacter or Bacteroides genomes or fragments thereof, have been tested for their ability to stimulate differentiation of naïve CD4+ and CD8+ T cells (Th0) to CD4+ and CD8+ activated T cells (Tact).

Described herein are a number of peptides and variants thereof, e.g., as shown in Tables 1-7.

TABLE 1

| Sequence | | |
|---|---|---|
| Identifier | Peptide Name | Amino Acid Sequence |
| SEQ ID NO: 1 | SG-3-00802 | MKVRPSVKPMCEKCKIIRRKGRVMVICENPKHKQRQG |

A variant of SEQ ID NO:1 according to the present disclosure can be a peptide with 1, 2, 3, 4, 5, 6, 7, 8, 9, 10, 11, 12, 13, 14, or 15 amino acid modifications relative to SEQ ID NO:1. In some embodiments, the variant can be at least 50%, 55%, 60%, 65%, 70%, 75%, 80%, 85%, 90%, 91%, 92%, 93%, 94%, 95%, 96%, 97%, or 98% identical to SEQ ID NO:1. Examples of amino acid modifications with respect to the amino acid sequence set forth in SEQ ID NO:1, include, without limitation, amino acid substitutions, amino acid deletions, and amino acid insertions. In some embodiments, a peptide of the present disclosure has a deletion of 1, 2, 3, 4 or 5 N- or C-terminal residues of SEQ ID NO:1. Alternatively or additionally, there is an internal deletion of 1, 2, 3, 4, or 5 amino acids relative to SEQ ID NO: 1. Variants of SEQ ID NO: 1 can be 30-45 amino acids long (e.g., 36, 37, 38, or 39 amino acids long).

An amino acid substitution of SEQ ID NO:1 can be a conservative amino acid substitution. For example, conservative amino acid substitutions can be made by substituting one amino acid residue for another amino acid residue having a similar side chain.

In some embodiments, an amino acid substitution of SEQ ID NO:1 is a non-conservative amino acid substitution. Non-conservative amino acid substitutions can be made by substituting one amino acid residue for another amino acid residue having a dissimilar side chain.

In some embodiments, the methionine at position at 1 of SEQ ID NO:1 is substituted with another amino acid (e.g., any of the other naturally-occurring amino acids).

In some embodiments, the valine at position at 3 of SEQ ID NO:1 is substituted with another amino acid (e.g., any of the other naturally-occurring amino acids). In some embodiments, the valine at position at 3 of SEQ ID NO:1 is substituted with an amino acid selected from the group consisting of: I and T.

In some embodiments, the arginine at position at 4 of SEQ ID NO:1 is substituted with another amino acid (e.g., any of the other naturally-occurring amino acids). In some embodiments, the arginine at position at 4 of SEQ ID NO:1 is substituted with an amino acid selected from the group consisting of K and Q.

In some embodiments, the proline at position at 5 of SEQ ID NO:1 is substituted with another amino acid (e.g., any of the other naturally-occurring amino acids). In some embodiments, the proline at position at 5 of SEQ ID NO:1 is substituted with an amino acid selected from the group consisting of: S and A.

In some embodiments, the proline at position at 9 of SEQ ID NO:1 is substituted with another amino acid (e.g., any of the other naturally-occurring amino acids). In some embodiments, the proline at position at 9 of SEQ ID NO:1 is substituted with an amino acid selected from the group consisting of: T and K.

In some embodiments, the methionine at position at 10 of SEQ ID NO:1 is substituted with another amino acid (e.g., any of the other naturally-occurring amino acids). In some embodiments, the methionine at position at 10 of SEQ ID NO:1 is substituted with the amino acid I.

In some embodiments, the glutamic acid at position at 12 of SEQ ID NO:1 is substituted with another amino acid (e.g., any of the other naturally-occurring amino acids). In some embodiments, the glutamic acid at position at 12 of SEQ ID NO:1 is substituted with the amino acid D.

In some embodiments, the lysine at position at 13 of SEQ ID NO:1 is substituted with another amino acid (e.g., any of the other naturally-occurring amino acids). In some embodiments, the lysine at position at 13 of SEQ ID NO:1 is substituted with the amino acid Y.

In some embodiments, the lysine at position at 15 of SEQ ID NO:1 is substituted with another amino acid (e.g., any of the other naturally-occurring amino acids). In some embodiments, the lysine at position at 15 of SEQ ID NO:1 is substituted with the amino acid R.

In some embodiments, the valine at position at 16 of SEQ ID NO:1 is substituted with another amino acid (e.g., any of the other naturally-occurring amino acids). In some embodiments, the valine at position at 16 of SEQ ID NO:1 is substituted with the amino acid I.

In some embodiments, the lysine at position at 18 of SEQ ID NO:1 is substituted with another amino acid (e.g., any of the other naturally-occurring amino acids). In some embodiments, the lysine at position at 18 of SEQ ID NO:1 is substituted with the amino acid R.

In some embodiments, the lysine at position at 20 of SEQ ID NO:1 is substituted with another amino acid (e.g., any of the other naturally-occurring amino acids). In some embodiments, the lysine at position at 20 of SEQ ID NO:1 is substituted with an amino acid selected from the group consisting of: N and H.

In some embodiments, the arginine at position at 22 of SEQ ID NO:1 is substituted with another amino acid (e.g., any of the other naturally-occurring amino acids). In some embodiments, the arginine at position at 22 of SEQ ID NO:1 is substituted with an amino acid selected from the group consisting of K, H, S, and I.

In some embodiments, the valine at position at 23 of SEQ ID NO:1 is substituted with another amino acid (e.g., any of the other naturally-occurring amino acids). In some embodiments, the valine at position at 23 of SEQ ID NO:1 is substituted with the amino acid I.

In some embodiments, the methionine at position at 24 of SEQ ID NO:1 is substituted with another amino acid (e.g., any of the other naturally-occurring amino acids). In some embodiments, the methionine at position at 24 of SEQ ID NO:1 is substituted with an amino acid selected from the group consisting of R, A, and L.

In some embodiments, the valine at position at 25 of SEQ ID NO:1 is substituted with another amino acid (e.g., any of the other naturally-occurring amino acids). In some embodiments, the valine at position at 25 of SEQ ID NO:1 is substituted with the amino acid I.

In some embodiments, the glutamic acid at position at 28 of SEQ ID NO:1 is substituted with another amino acid (e.g., any of the other naturally-occurring amino acids). In some embodiments, the glutamic acid at position at 28 of SEQ ID NO:1 is substituted with an amino acid selected from the group consisting of Q, A, and T.

In some embodiments, the asparagine at position at 29 of SEQ ID NO:1 is substituted with another amino acid (e.g., any of the other naturally-occurring amino acids). In some embodiments, the asparagine at position at 29 of SEQ ID NO:1 is substituted with the amino acid E.

In some embodiments, the lysine at position at 31 of SEQ ID NO:1 is substituted with another amino acid (e.g., any of the other naturally-occurring amino acids). In some embodiments, the lysine at position at 31 of SEQ ID NO:1 is substituted with the amino acid R.

In some embodiments, the lysine at position at 35 of SEQ ID NO:1 is substituted with another amino acid (e.g., any of the other naturally-occurring amino acids). In some embodiments, the lysine at position at 35 of SEQ ID NO:1 is substituted with the amino acid R.

In some embodiments, none of the amino acids of SEQ ID NO:1 are substituted with cysteine.

In some embodiments, a peptide described herein has an amino acid sequence selected from Table 2 (e.g., SEQ ID NOs 1-45), where Xx represents any naturally-occurring amino acid.

In some embodiments, X₁ of SEQ ID NO:45 is the amino acid M.

In some embodiments, X₃ of SEQ ID NO:45 is an amino acid selected from the group consisting of: V, I, and T.

In some embodiments, X₄ of SEQ ID NO:45 is an amino acid selected from the group consisting of: R, K, and Q.

In some embodiments, X₅ of SEQ ID NO:45 is an amino acid selected from the group consisting of: P, S, and A.

In some embodiments, X₉ of SEQ ID NO:45 is an amino acid selected from the group consisting of: P, T, and K.

In some embodiments, X₁₀ of SEQ ID NO:45 is an amino acid selected from the group consisting of: M and I.

In some embodiments, X₁₂ of SEQ ID NO:45 is an amino acid selected from the group consisting of: E and D.

In some embodiments, X₁₃ of SEQ ID NO:45 is an amino acid selected from the group consisting of: K and Y.

In some embodiments, X₁₅ of SEQ ID NO:45 is an amino acid selected from the group consisting of: K and R.

In some embodiments, X₁₆ of SEQ ID NO:45 is an amino acid selected from the group consisting of: V and I.

In some embodiments, X₁₈ of SEQ ID NO:45 is an amino acid selected from the group consisting of: K and R.

In some embodiments, X₂₀ of SEQ ID NO:45 is an amino acid selected from the group consisting of: K, N, and H.

In some embodiments, X₂₂ of SEQ ID NO:45 is an amino acid selected from the group consisting of: R, K, H, S, and I.

In some embodiments, X₂₃ of SEQ ID NO:45 is an amino acid selected from the group consisting of: V and I.

In some embodiments, X₂₄ of SEQ ID NO:45 is an amino acid selected from the group consisting of: M, R, A, and L.

In some embodiments, X₂₅ of SEQ ID NO:45 is an amino acid selected from the group consisting of: V and I.

In some embodiments, X₂₈ of SEQ ID NO:45 is substituted with an amino acid selected from the group consisting of: E, Q, A, and T.

In some embodiments, X₂ of SEQ ID NO:45 is substituted with an amino acid selected from the group consisting of: N and E.

In some embodiments, X₃₁ of SEQ ID NO:45 is substituted with an amino acid selected from the group consisting of K and R.

In some embodiments, X₃₅ of SEQ ID NO:45 is substituted with an amino acid selected from the group consisting of K and R.

Specific exemplary amino acid sequences that include the above mentioned amino acid sequences are listed in Table 2 and Table 3 (e.g., SEQ ID NOs 1-45, 47-50).

TABLE 2

| Sequence | |
|---|---|
| Identifier | Amino Acid Sequence |
| SEQ ID NO: 1 | MKVRPSVKPMCEKCKIIRRKGRVMVICENPKHKQRQG |
| SEQ ID NO: 2 | MKVRPSVKPICEKCKIIRRKGRVMVICENPKHKQKQG |
| SEQ ID NO: 3 | MKVRPSVKPMCEKCKIIKRKGKVMVICENPKHKQRQG |
| SEQ ID NO: 4 | MKVRPSVKPMCEKCKIIKRKGKVMVICENPKHKQKQG |
| SEQ ID NO: 5 | MKVRPSVKPICEKCKIIRRKGRVMVICQNPKHKQKQG |
| SEQ ID NO: 6 | MKVRPSVKPICEKCKIIKRKGRVMVICENPKHKQKQG |
| SEQ ID NO: 7 | MKVRPSVKPMCEKCKVIKRKGKVMVICENPKHKQRQG |
| SEQ ID NO: 8 | MKVRPSVKPMCEKCKVIKRKGRVMVICENPKHKQKQG |
| SEQ ID NO: 9 | MKVRPSVKPICEKCKVIRRKGRVMVICENPKHKQKQG |
| SEQ ID NO: 10 | MKVKPSVKPICEKCKIIKRKGRVMVICENPKHKQKQG |
| SEQ ID NO: 11 | MKVRPSVKPMCEKCKVIKRKGKVMVICENPKHKQKQG |
| SEQ ID NO: 12 | MKVRPSVKKICEKCKIIKRKGRVMVICENPKHKQKQG |
| SEQ ID NO: 13 | MKIRPSVKPMCEKCRIIKRKGRVMVICENPKHKQKQG |
| SEQ ID NO: 14 | MKVRPSVKPICEKCKVIRRKGKVMVICENPKHKQKQG |
| SEQ ID NO: 15 | MKVRPSVKPICEKCKIIKRKGKVMVICENPKHKQKQG |
| SEQ ID NO: 16 | MKVRPSVKPICEKCKIIRRKGKVMVICQNPKHKQKQG |
| SEQ ID NO: 17 | MKVRPSVKPICEKCKVIKRKGRVMVICENPKHKQKQG |
| SEQ ID NO: 18 | MKVKPSVKTICEKCKIIRRKGRVMVICENPKHKQKQG |
| SEQ ID NO: 19 | MKVRPSVKPICEKCKVIKRKGKVMVICENPKHKQRQG |
| SEQ ID NO: 20 | MKVRPSVKPICDKCRIIKRKGRVMVICENPKHKQRQGN |
| SEQ ID NO: 21 | MKVKPSVKPICEKCKVIRRKGRVMVICQNPKHKQRQG |
| SEQ ID NO: 22 | MKVKPSVKTICEKCKIIKRKGRVMVICENPKHKQKQG |
| SEQ ID NO: 23 | MKVQPSVKKICEKCKIIKRKGRVMVICENPKHKQKQG |
| SEQ ID NO: 24 | MKVRSSVKPICEKCKIIRRKGSIRVICENPKHKQRQG |
| SEQ ID NO: 25 | MKVKPSVKPICEKCKVIKRKGRVMVICQNPKHKQRQG |
| SEQ ID NO: 26 | MKVRSSVKPICEKCKIIKRKGRIRVICENPKHKQRQG |
| SEQ ID NO: 27 | MKVRPSVKPICEKCKVIKRKGKVMVICENPKHKQKQG |
| SEQ ID NO: 28 | MKVKPSVKKICEKCKIIKRKGRVMVICENPKHKQKQG |
| SEQ ID NO: 29 | MKVRPSVKPICEKCKVIKRKGKVMVICQNPKHKQRQG |
| SEQ ID NO: 30 | MKVKPSVKTICEKCKIIRRKGRVMIICENPKHKQKQG |
| SEQ ID NO: 31 | MKVRPSVKPICEKCKVIKRKGHVMVICENPKHKQKQG |
| SEQ ID NO: 32 | MKVRPSVKPICDKCRVIKRKGRVMVICENPKHKQRQG |
| SEQ ID NO: 33 | MKVRSSVKPICEKCKIIRRKGSIRVICENPKHKQRQDRH |
| SEQ ID NO: 34 | MKVRPSVKPICEYCKVIRRNGRVMVICPTNPKHKQRQG |
| SEQ ID NO: 35 | MKVRSSVKPICEKCKIIKRKGSIRVICENPKHKQRQG |
| SEQ ID NO: 36 | MKVKPSVKPICEKCKVIKRKGRVMIICANPKHKQRQG |
| SEQ ID NO: 37 | MKVKPSVKTICEKCKIIKRKGRVMIICENPKHKQKQG |
| SEQ ID NO: 38 | MKTRSSVKPMCDKCKVIKRKGRVAVICENPKHKQRQG |
| SEQ ID NO: 39 | MKVRPSVKPMCDKCKVIKRKGKVMVICQEPKHKQRQG |
| SEQ ID NO: 40 | KVRSSVKPICEKCKVIKRKGIVRVICENPKHKQRQG |
| SEQ ID NO: 41 | MKVRASVKPICDKCKVIKRKGIVRVICENPKHKQRQG |
| SEQ ID NO: 42 | MKVRPSVKKMCDKCKIIRRHGKILVICENPRHKQRQG |
| SEQ ID NO: 43 | MKVRPSVKKMCDKCKVIKRKGKILVICENPKHKQRQG |
| SEQ ID NO: 44 | MKVRSSVKPICEKCKVIKRKGSVRIICENPKHKQRQG |
| SEQ ID NO: 45 | X₁KX₃X₄X₅SVKX₉X₁₀CX₁₂X₁₃CX₁₄X₁₅X₁₆IX₁₈RX₂₀GX₂₂X₂₃X₂₄X₂₅IX₂₇ |
| | X₂₈X₂₉PX₃₁HKQX₃₅QX₃₇ |

TABLE 3

| Sequence | | |
|---|---|---|
| Identifier | Peptide Description | Amino Acid Sequence |
| SEQ ID | SG-3-06683, retro-inverso | GQRQKHKPNECIVMVRGKRRIIKCKECMPKVSPR |
| NO: 46 | peptide variant of SEQ ID NO: | VKM |
| | 1 with D-amino acids | |
| SEQ ID | SG-3-01677 | MKVRSSVKPICEKCKVIKRKGAIRVICENPKHKQR |
| NO: 47 | | QG |
| SEQ ID | SG-3-01797 | MKVRPSVKKMCDKCKIIRRHGKVLVICENPRHKQ |
| NO: 48 | | RQG |
| SEQ ID | SG-3-03562 | MKVRPSVKPMCDKCRVIKRKGRVMIICENPKHKQ |
| NO: 49 | | RQG |
| SEQ ID | SG-3-03785 | MKVRPSVKPMCEKCKIIKRKGKVMVICENPKHKQ |
| NO: 50 | | RQG |
| SEQ ID | SG-3-05021 | LGKTDWIEKYFKVKKEKIDKMQRFLQG |
| NO: 51 | | |

Peptides disclosed herein can be modified according to the methods known in the art for producing peptidomimetics. See, e.g., Qvit et al., Drug Discov Today. 2017 February; 22(2): 454-462; Farhadi and Hashemian, Drug Des Devel Ther. 2018; 12: 1239-1254; Avan et al., Chem. Soc. Rev., 2014, 43, 3575-3594; Pathak, et al., Indo American Journal of Pharmaceutical Research, 2015. 8; Kazmierski, W. M., ed., Peptidomimetics Protocols, Human Press (Totowa NJ 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “inverso” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “retro” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetic include replacing one or more amino acid residues in a peptide with a chemically distinct but recognized functional analog of the amino acid, i.e., an artificial amino acid analog. Artificial amino acid analogs include β-amino acids, β-substituted β-amino acids (“(β³-amino acids”), phosphorous analogs of amino acids, such as α-amino phosphonic acids and α-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. Exemplary retro-inverso targeting peptidomimetics include GQRQKHKPNECIVMVRGKRRIIKCKECMPKVSPR VKM (SG-3-06683, SEQ ID NO: 46), wherein the sequences include all D-amino acids. These sequences can be modified, e.g., by biotinylation of the amino terminus and amidation of the carboxy terminus.

Peptides can contain either L-amino acids, D-amino acids, or a combination of L-amino acids and D-amino acids. In some embodiments, a peptide having the amino acid sequence of SEQ ID NO: 46 (SG-3-06683) contains D-amino acids and includes reverse sequences compared to SEQ ID NO: 1 (SG-3-00802). Exemplary amino acid sequences that include the above mentioned amino acid sequences are listed in Table 3 (e.g., SEQ ID NO: 46-51).

In some embodiments, provided herein are peptides comprising or consisting of the amino acid sequence set forth in SEQ ID NO:46 (See Table 3—SG-3-06683) or SEQ ID NO:51 (See Table 3—SG-3-05021).

A variant of SEQ ID NO:46 or 51 according to the present disclosure can be a peptide with 1, 2, 3, 4, 5, 6, 7, 8, 9, 10, 11, 12, 13, 14, or 15 amino acid modifications relative to SEQ ID NO:46 or 51. In some embodiments, the variant can be at least 50%, 55%, 60%, 65%, 70%, 75%, 80%, 85%, 90%, 91%, 92%, 93%, 94%, 95%, 96%, 97%, 98%, 99%, or 99.5% identical to SEQ ID NO:46 or 51. Examples of amino acid modifications with respect to the amino acid sequence set forth in SEQ ID NO:46 or 51, include, without limitation, amino acid substitutions, amino acid deletions, and amino acid insertions. In some embodiments, a peptide of the present disclosure has a deletion of 1, 2, 3, 4 or 5 N- or C-terminal residues of SEQ ID NO:46 or 51. Alternatively or additionally, there is an internal deletion of 1, 2, 3, 4, or 5 amino acids relative to SEQ ID NO:46 or 51.

An amino acid substitution of SEQ ID NO:46 or 51 can be a conservative amino acid substitution. For example, conservative amino acid substitutions can be made by substituting one amino acid residue for another amino acid residue having a similar side chain.

In some embodiments, an amino acid substitution of SEQ ID NO:46 or 51 is a non-conservative amino acid substitution. Non-conservative amino acid substitutions can be made by substituting one amino acid residue for another amino acid residue having a dissimilar side chain.

Any of the amino acids described herein contain mutations, e.g. at least one mutation compared to the wild-type (e.g., naturally occurring) peptide, protein, or amino acid to make a variant peptide. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some embodiments, an amino acid substitution is a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

In addition to or instead of mutations, any peptide described herein (e.g., a peptide having the amino acid sequence of SEQ ID NO: 1, 46, or 51 or a variant thereof, e.g., as shown in Tables 1-7) can be modified. For example, an acetyl (Ac) and/or or an amide group can be added at the C- and/or N-terminus of the peptide. In some embodiments, the peptide is “cyclized,” referring to a reaction in which one part of a polypeptide or peptide molecule becomes linked to another part of the polypeptide or peptide molecule to form a closed ring, such as by forming a disulfide bridge or other similar bond. In some embodiments, a peptide described herein (e.g., a peptide having the amino acid sequence of SEQ ID NO: 1, 46, or 51 or a variant thereof) is linked to another molecule, e.g., protein (e.g., BSA or Fc domain) or stabilizing group such as a PEG molecule, by a linker. A “linker moiety,” as used herein, refers broadly to a chemical structure that is capable of linking or joining together two peptide monomer subunits to form a dimer.

In some embodiments, a peptide of the present disclosure can be modified to increase the solubility of the peptide in an aqueous solution, relative to the unmodified peptide. In some embodiments, a peptide of the present disclosure can be modified to decrease the solubility of the peptide in an aqueous solution, relative to the unmodified peptide. In some embodiments, a peptide of the present disclosure can be modified to increase the solubility of the peptide in a polar solvent, relative to the unmodified peptide. In some embodiments, a peptide of the present disclosure can be modified to decrease the solubility of the peptide in a polar solvent, relative to the unmodified peptide. In some embodiments, a peptide of the present disclosure can be modified to increase the solubility of the peptide in a non-polar solvent, relative to the unmodified peptide. In some embodiments, a peptide of the present disclosure can be modified to decrease the solubility of the peptide in a non-polar solvent, relative to the unmodified peptide.

In some embodiments, a peptide of the present disclosure can be modified to increase the net charge of the peptide at human physiological pH, relative to the unmodified peptide at human physiological pH. In some embodiments, a peptide of the present disclosure can be modified to decrease the net charge of the peptide at human physiological pH, relative to the unmodified peptide at human physiological pH.

In some embodiments, a peptide described herein can have a post-translational modification (PTM). Protein PTMs (e.g., peptide PTMs) occur in vivo and can increase the functional diversity of the proteome by the covalent addition of functional groups or proteins, proteolytic cleavage of regulatory subunits or degradation of entire proteins. Isolated peptides prepared according to the present disclosure can undergo one or more PTMs in vivo or in vitro. The type of modification(s) depends on host cell in which the peptide is expressed and includes but is not limited to phosphorylation, glycosylation, ubiquitination, nitrosylation (e.g., S-nitrosylation), methylation, acetylation (e.g., N-acetylation), lipidation (myristoylation, N-myristoylation, S-palmitoylation, farnesylation, S-prenylation, S-palmitoylation) and proteolysis can influence aspects of normal cell biology and pathogenesis. The peptides as disclosed herein can comprise one or more the above recited post-translational modifications.

In some embodiments, a peptide described herein (e.g., peptide having the amino acid sequence of SEQ ID NO: 1, 46, or 51 or a variant thereof) modulates the activity of a CXCR3 protein, a CXCR4 protein, or a CCR9 protein. In some cases, a peptide described herein (e.g., peptide having the amino acid sequence of SEQ ID NO: 1, 46, or 51 or a variant thereof) increases the activity of a CXCR3 protein or a CCR9 protein. In some cases, a peptide described herein (e.g., a peptide having the amino acid sequence of SEQ ID NO: 1, 46, or 51, or a variant thereof) decreases the activity of CXCR4. See tables 9-11 for additional information about the activity of CXCR3, CXCR4, and CCR9.

Activity of an anti-tumor or protein (e.g., a peptide of SEQ ID NO: 1, 46, or 51, or variants thereof) is measured in a beta-arrestin reporter cell assay that monitors the activity of a GPCR, including CXCR3, CXCR4, and CCR9.

In some embodiments, a peptide described herein (e.g., a peptide having the amino acid sequence of SEQ ID NO: 1, 46, or 51 or a variant thereof) binds to a CCR9 protein, a CXCR3 protein, or a CXCR4 protein. In some embodiments, a peptide described herein (e.g., a peptide having the amino acid sequence of SEQ ID NO: 1, 46, or 51 or a variant thereof) binds to a CXCR3 protein.

### Methods of Use

Provided herein are methods for treating subjects in need thereof (e.g., subjects having an immunological disease and/or cancer) using one or more peptides described herein (e.g., a peptide having the amino acid sequence of SEQ ID NO: 1, 46, or 51 or a variant thereof, e.g., as shown in Tables 1-7). Immunological diseases and cancers that can be treated using a peptide as described herein (e.g., a peptide having the amino acid sequence of SEQ ID NO: 1, 46, or 51 or a variant thereof) can include diseases that are associated with inflammatory immune responses. In some embodiments, a peptide described herein (e.g., a peptide having the amino acid sequence of SEQ ID NO: 1, 46, or 51 or a variant thereof) modulates immunoregulatory cells, including but not limited to T cells, effector T cells and dendritic cells. In some embodiments, the subject has a T-cell mediated disease. In some embodiments, the immunological disease and/or cancer includes those that are characterized by infiltration of inflammatory cells into a tissue, stimulation of T cell proliferation, inhibition of T cell proliferation, increased or decreased vascular permeability, or the inhibition thereof.

In some embodiments, the method uses one or more recombinant hosts. In some embodiments, the method uses one or more pharmaceutical compositions. In some embodiments, the method uses one or more nucleic acid constructs.

In some embodiments, administration of a peptide described herein (e.g., a peptide having the amino acid sequence of SEQ ID NO: 1, 46, or 51 or a variant thereof) can prevent, reduce the severity of, or eliminate at least one symptom, of the disease or condition in the subject. The subject may be an animal. The subject may be a mammal. The subject may be a human subject.

In some embodiments, the disease or condition is cancer, e.g., any of the cancers described herein. In some embodiments, the disease or condition is autoimmune thyroiditis. In some embodiments, the disease or conditions is Hodgkin's lymphoma.

In some embodiments of the methods provided herein, a peptide described herein (e.g., a peptide having the amino acid sequence of SEQ ID NO: 1, 46, or 51 or a variant thereof) can modulate the production of at least one cytokine in the subject. In some embodiments of the methods provided herein, a peptide described herein (e.g., a peptide having the amino acid sequence of SEQ ID NO: 1, 46, or 51 or a variant thereof) can induce or increase the production of at least one pro-inflammatory cytokine (e.g., TNF-α and/or IL-23) by an immune cell (e.g., in an immune cell in a subject administered the peptide). In some embodiments of the methods provided herein, the peptide can suppress (e.g., prevent, inhibit, or decrease the production of) at least one anti-inflammatory cytokine (e.g., IL-10) by an immune cell (e.g., in an immune cell in a subject administered the peptide). Pro-inflammatory cytokines can include TNF-α, IL-17, IL-1β, IL-2, IFN-γ, IL-6, IL-12, IL-25, IL-33, IL-8, MCP-1, MIP-3α, CXCL1, and IL-23. Anti-inflammatory cytokines can include IL-4, IL-10, IL-13, IFN-α, and TGF-β.

In some embodiments, treatment with a peptide described herein can induce naïve T cells to differentiate. In some embodiments, treatment with a peptide described herein (e.g., a peptide having the amino acid sequence of SEQ ID NO: 1, 46, or 51 or a variant thereof) can induce production of IFN-γ and IL-4 from T cells. In some embodiments, treatment with a peptide described herein (e.g., a peptide having the amino acid sequence of SEQ ID NO: 1, 46, or 51 or a variant thereof) can induce maintained IFN-γ production from a variety of T cell subsets. In some embodiments, a peptide described herein (e.g., a peptide having the amino acid sequence of SEQ ID NO: 1, 46, or 51 or a variant thereof) increases Th1 activation in a subject administered the peptide. In some embodiments, a peptide described herein (e.g., a peptide having the amino acid sequence of SEQ ID NO: 1, 46, or 51 or a variant thereof) increases dendritic cell maturation, in a subject administered the peptide. In some embodiments, a peptide described herein increases the number of antigen-presenting T cell-priming cells. In some embodiments, a peptide described herein (e.g., a peptide having the amino acid sequence of SEQ ID NO: 1, 46, or 51 or a variant thereof) can increase conversion of antigens into immunogens. In some embodiments, a peptide described herein (e.g., a peptide having the amino acid sequence of SEQ ID NO: 1, 46, or 51 or a variant thereof) increases CD70 expression in a subject administered the peptide. In some embodiments, a peptide described herein (e.g., a peptide having the amino acid sequence of SEQ ID NO: 1, 46, or 51 or a variant thereof) increases the clonal expansion of T_{eff}, in a subject administered the peptide.

In some embodiments, a peptide described herein increases the number to T cells in a T cell subtype selected from the group consisting of CD4⁺CD25⁺, CD4⁺PD-1⁺, CD4⁺ICOS⁺, CD4⁺OX40⁺, CD8⁺CD25⁺, CD8⁺PD-1⁺, CD8⁺ICOS⁺, and CD8⁺OX40⁺ in the subject. In some embodiments, a peptide described herein increases secretion of one or more cytokines selected from the group consisting of IFN-γ, IL-2, IL-10 and TNF-α in the subject.

In some embodiments of the methods provided herein, treatment with a peptide described herein (e.g., a peptide having the amino acid sequence of SEQ ID NO: 1, 46, or 51 or a variant thereof) modulates activity of a CXCR3 protein, a CXCR4 protein, and/or a CCR9 protein. In some embodiments of the methods provided herein, treatment with a peptide described herein (e.g., a peptide having the amino acid sequence of SEQ ID NO: 1, 46, or 51 or a variant thereof) increases activity of a CXCR3 protein and/or a CCR9 protein. In some embodiments of the methods provided herein, treatment with a peptide described herein decreases activity of a CCR9 protein. In some embodiments of the methods provided herein, treatment with a peptide described herein (e.g., a peptide having the amino acid sequence of SEQ ID NO: 1, 46, or 51 or a variant thereof) results in the peptide binding to a CCR9 protein, a CXCR3 protein, or a CXCR4 protein. In some embodiments of the methods provided herein, treatment with a peptide described herein results in the peptide binding to a CXCR3 protein.

Also provided herein are method for increasing the response to an immunotherapy in a subject in need that includes administering to the subject a peptide described herein (e.g., a peptide having the amino acid sequence of SEQ ID NO: 1, 46, or 51 or a variant thereof), a recombinant host described herein, or a pharmaceutical composition described herein.

Any of the methods of treatment described herein can include administering a therapy to the identified subject. The therapy can include one or more peptides described herein, one or more pharmaceutical compositions described herein, and/or one or more pharmaceutical compositions described herein.

In some embodiments, additional treatment(s) of cancer can include chemotherapeutic agents. Non-limiting examples of chemotherapeutic agents include carboplatin, cisplatin, gemcitabine, methotrexate, paclitaxel, pemetrexed, lomustine, temozolomide, dacarbazine, and combinations thereof. Non-limiting examples of targeted therapies include afatinib dimaleate, bevacizumab, cetuximab, crizotinib, erlotinib, gefitinib, sorafenib, sunitinib, pazopanib, everolimus, dabrafenib, aldesleukin, interferon alfa-2b, peginterferon alfa-2b, trametinib, vemurafenib, and combinations thereof. Non-limiting examples of an immunotherapy include an immune checkpoint inhibitor (e.g,. ipilimumab, nivolumab, pembrolizumab, atezolizumab, avelumab, durvalumab, cemiplimab, and combinations thereof); co-stimulatory immune checkpoint agent (e.g., IBI101, utomilumab, MEDI1873, and combinations thereof); and a cell therapy (e.g., a CAR T cell therapy). In some examples, the therapy is a CAR T cell therapy.

In some embodiments, a prebiotic and/or probiotic can be administered in combination with a composition comprising a peptide as described herein. Non-limiting examples of a probiotic include one of more of Bifidobacteria (e.g., B. animalis, B. breve, B. lactis, B. longum, or B. infantis), Lactobacillus (e.g., L. acidophilus, L. reuteri, L. bulgaricus, L. lactis, L. casei, L. rhamnosus, L. plantarum, L. paracasei, or L. delbreuckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nutr. 137:2580-2584).

In some embodiments, methods provided herein can include administering a peptide or pharmaceutical composition thereof described herein to the subject at least once per day. For example, the peptide or pharmaceutical composition thereof can be administered two, three, four, or more times per day. In some embodiments, an effective amount of the peptide or pharmaceutical composition thereof is administered in one dose, e.g., once per day. In some embodiments, an effective amount of the peptide or pharmaceutical composition thereof is administered in more than one dose, e.g., more than once per day. In some embodiments, the method comprises administering the peptide, or pharmaceutical composition thereof to the subject daily, every other day, every three days, or once a week.

An immunotherapy can include an immune checkpoint inhibitor and/or a co-stimulatory immune checkpoint agent. Non-limiting examples of immune checkpoint inhibitors include inhibitors that target CTLA-4 (cytotoxic T-lymphocyte-associated protein 4) such as ipilimumab (YERVOY®); PD-1 (Programmed cell death protein 1) such as pembrolizumab (KEYTRUDA®), nivolumab (OPDIVO®), or cemiplimab (LIBTAYO®); PD-L1 (Programmed death-ligand 1) such as atezolizumab (TECENTRIQ®), avelumab (BAVENCIO®), or durvalumab (IMFINZI®); BTLA (B and T lymphocyte attenuator); LAG-3 (Lymphocyte Activation Gene 3) such as IMP701 (LAG525); A2AR (Adenosine A2a receptor) such as CPI-444; TIM-3 (T-cell immunoglobulin and mucin domain-3) such as MBG453; B7-H3 (B7 homolog 3; also known as CD276) such as enoblituzumab; VISTA (V-domain Ig suppressor of T cell activation) such as JNJ-61610588; and IDO (Indole amine 2,3-dioxygenase) such as indoximod. See, for example, Marin-Acevedo, et al., J Hematol Oncol. 11: 39 (2018). Non-limiting examples of co-stimulatory immune checkpoint agents include agents that target OX40 such as IBI101; 4-1BB such as utomilumab (PF-05082566); and GITR such as MEDI1873.

Immunotherapies can modulate (e.g., increase or decrease) expression or activity of CTLA-4, PD-1, PD-L1, BTLA, LAG-3, A2AR, TIM-3, B7-H3, VISTA, IDO, OX40, 4-1BB, TIGIT, and/or GITR. For example, immunotherapies, including immunotherapies such as any of the peptides described herein, can inhibit or decrease the activity of CTLA-4, PD-1, PD-L1, BTLA, LAG-3, A2AR TIM-3, GITR, B7-H3, VISTA, IDO, GITR and/or TIGIT; or increase the activity of OX40 or 4-1BB.

While immunotherapies such as those described herein have largely been effective, many subjects do not respond to immune checkpoint inhibitors (see, e.g., Humphries and Daud. Hum Vaccin Immunother. 2018; 14(9): 2178-2182). The gut microbiome may be one of the factors that determines the efficacy of immune checkpoint treatment and whether a subject responds to such treatment.

The level of expression of genes can be measured with a variety of RNA-based techniques that are known in the art including reverse-transcription polymerase chain reaction (RT-PCR), quantitative RT-PCR (qRT-PCR), global transcriptomics, or RNA-seq.

The level of activity of an enzymes can be measured with a variety of protein-based methods that are known in the art including reporter assays, Western blots, ELISA assays, mass spectrometry, or global proteomic analysis.

Any of the methods described herein can include detecting the level of one or more bacterial species, RNA transcripts, protein activity, or flux though a metabolic pathway in a sample from the subject. Detecting levels of bacterial species, RNA transcripts, protein activity or flux can include any of the methods of detection described above.

### T Cells

A critical step in mounting an immune response in mammals is the activation of an appropriate set of T cells which can recognize an antigen associated with a disease or disorder. T cells can differentiate into helper, regulatory, cytotoxic or memory T cells. CD4+ and CD8+ T cells make up the majority of T cells. CD4+ helper T cells recognize MHC-II restricted exogenous antigens that have been captured and processed in the cellular endosomal pathway in antigen presenting cells, such as dendritic cells (DCs), then complexed onto the MHC-II in the Golgi compartment to form an antigen-MHC-II complex. This complex, expressed on the cell surface, can induce activation of CD4+ effector T cells.

CD4+ T cells can be activated and differentiated into distinct effector subtypes including T-helper 1 (Th1), T-helper 2 (Th2), T-helper 17 (Th17), follicular helper T cell (Tfh), induced T-regulatory cells (iTreg) and regulatory type 1 cells (Tr1). CD4+ T cells (Th cells) produce interleukins which in turn help to activate other arms of the immune system. For example, Th cells produce interleukin-4 (IL-4) and IL-5, which aid B cells in producing antibodies; IL-2 which activates CD4+ and CD8+ T cells. Interleukin-12 (IL12) and interferon γ (IFNγ) are critical cytokines which initiate the downstream signaling cascade to develop Th1 cells. The IL12, in turn, induces natural killer cells (NK) to produce IFNγ. Since Th cells that recognize MHC-II restricted antigens play a central role in the activation and clonal expansion of cytotoxic T cells, macrophages, natural killer (NK) cells, and B cells, the initial event of activating the helper T cells in response to an antigen is crucial for the induction of an effective immune response directed against that antigen.

CD8+ T cells (cytotoxic T lymphocytes) are important for immune defense against intracellular pathogens and for tumor surveillance. CD8+ cells are activated when the desired protein/peptide is routed through the cell in such a manner so as to be presented on the cell surface as a processed protein/peptide, which is complexed with MHC-I antigens. CD8+ cytotoxic T cells destroy infected target cells though the release of perform, granzymes, and granulysin.

Regulatory T cells (T_{regs}) function primarily to suppress potentially deleterious activities of Th cells. T_{regs} may express a member of the FOX protein family, forkhead box P3 (FOXP3), which functions as a master regulator of the regulatory pathway in the development and function of regulatory T cells. T_{regs} are often involved in dialing down the immune response. In the instance of cancer, an excess of T_{reg} activity can prevent the immune system from destroying cancer cells.

### Dendritic Cells

In addition to the critical roles that T cells play in the immune response, dendritic cells (DCs) are equally important. DCs are professional antigen-presenting cells that process antigen material and present it on the cell surface to T cells. DCs having a key regulatory role in the maintenance of tolerance to self-antigens and in the activation of innate and adaptive immunity (Banchereau et al., 1998, Nature 392:245-52; Steinman et al., 2003, Annu. Rev. Immunol. 21:685-711).

DCs are derived from hematopoietic bone marrow progenitor cells, which initially transform into immature dendritic cells. Immature dendritic cells constantly sample the surrounding environment for pathogens, which is done through pattern recognition receptors such as the toll-like receptors (TLRs). Antigen-presenting cells (APCs), such as DCs and macrophages, play important roles in the activation of innate and adaptive immunity as well as in the maintenance of immunological tolerance.

When DCs encounter pro-inflammatory stimuli such as microbial products, the maturation process of the cell is initiated by up-regulating cell surface expressed antigenic peptide-loaded MHC molecules and co-stimulatory molecules. Following maturation and homing to local lymph nodes, DCs establish contact with T cells by forming an immunological synapse, where the T cell receptor (TCR) and co-stimulatory molecules congregate in a central area surrounded by adhesion molecules (Dustin et al., 2000, Nat. Immunol. 1:23-9). Once activated in the presence of DCs, e.g., CD8+ T cells can autonomously proliferate for several generations and acquire cytotoxic function without further antigenic stimulation (Kaech et al., 2001, Nat. Immunol. 2:415-22; van Stipdonk et al., 2001, Nat. Immunol. 2:423-9). It has therefore been proposed that the level and duration of peptide-MHC complexes (signal 1) and co-stimulatory molecules (signal 2) provided by DCs are essential for determining the magnitude and fate of an antigen-specific T cell response (Lanzavecchia et al., 2001, Nat. Immunol. 2:487-92; Gett et al., 2003, Nat. Immunol. 4:355-60).

DCs use TLRs, which recognize conserved microbial structures such as lipopolysaccharide (LPS), to promote DC maturation by activating the nuclear factor-κB (NF-κB) signaling pathway (Akira et al., 2004, Nat. Rev. Immunol. 4:499-51 1). Efforts to induce immunization to tumors have attempted to promote DC maturation and co-stimulation as a means of enhancing antitumor immunity.

Much attention has also been focused on pro-inflammatory signaling but less is known about the mechanisms that suppress and resolve inflammation. The magnitude and duration of TLR-initiated immune responses is dictated by the strength and duration of proinflammatory signaling and by the regulation of signal transduction pathways. Since TLR-induced activation of the transcription factor NF-κB is essential for the transcription of a large number of proinflammatory genes, multiple mechanisms are utilized to negatively regulate TLR signaling at multiple levels for the protection of subjects from excessive immune responses such as septic shock and for maintaining immune homeostasis in situations of chronic microbial exposure such as the intestinal microenvironment.

### Cytokines

Cytokines are small secreted proteins released by cells that have a specific effect on the interactions and communications between cells. Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). There are both pro-inflammatory cytokines and anti-inflammatory cytokines. Zhang et al., “Cytokines, Inflammation and Pain,” Int. Anesthesiol. Clin. , Vol. 45(2):27-37 (Spring 2007). Cytokines generally stimulate proliferation or differentiation of cells of the hematopoietic lineage or participate in the immune and inflammatory response mechanisms of the body.

Cytokines are critically involved in the regulation of multiple immune cell functions (Curtsinger et al., 2003, J. Exp. Med. 197:1141-51; Valenzuela et al., 2002, J. Immunol. 169:6842-9). As noted above, various immune cell phenotypes are characterized in terms of cytokines which they secrete. Cytokines are often classified as either pro- or anti-inflammatory.

The interleukins are a family of cytokines that mediate immunological responses. Central to an immune response is the T cell, which produces many cytokines and plays a role in adaptive immunity to antigens. Cytokines produced by the T cell have been classified as type 1 and type 2 (Kelso et al., 1998. Immun. Cell Biol. 76:.00-317). The type 1 cytokines include IL-2, IFN-γ, LT-α, and are involved in inflammatory responses, viral immunity, intracellular parasite immunity, and allograft rejection. Type 2 cytokines include IL-4, IL-5, IL-6, IL-10, and IL-13, and are involved in humoral responses, helminth immunity, and allergic response.

### Pro-Inflammatory Cytokines

Pro-inflammatory cytokines are cytokines that are important in cell signaling and promote systemic inflammation. They are produced predominantly by activated macrophages and are involved in the upregulation of inflammatory reactions. Pro-inflammatory cytokines arise from genes that code for the translation of small mediator molecules that induce a response after upregulation. Interleukin-1 (IL-1), IL-2, IL-6, IL-12, IL-17, IL-18, IL-23, CD40L, tumor necrosis factor (TNF) such as TNF-α, gamma-interferon (IFN-gamma), granulocyte-macrophage colony stimulating factor, MCP-1, TNF-related apoptosis-inducing ligand, RANK-ligand, and TALL-1/BAFF are well characterized as pro-inflammatory cytokines. Inflammation is characterized by an interplay between pro- and anti-inflammatory cytokines. In some embodiments, administration of the peptides of the present disclosure is accompanied by an increase in pro-inflammatory cytokines.

### Anti-Inflammatory Cytokines

Anti-inflammatory cytokines are a series of immunoregulatory molecules that control the pro-inflammatory cytokine response. These molecules thus modulate and help to decrease the pro-inflammatory response created by pro-inflammatory cytokines. IL-4, IL-10, IL-13, IFN-α (IFN), and transforming growth factor-beta (TGF-β) are recognized as anti-inflammatory cytokines. In some embodiments, administration of the peptides of the present disclosure is accompanied by a decrease of anti-inflammatory cytokines.

It is understood that there is a strong interplay with respect to the effects of cytokines. For example, the pro-inflammatory activity of one cytokine can be attenuated or eliminated by the anti-inflammatory activity of another.

### Target Proteins

Provided herein are method of modulating the activity of one or more target proteins. In some embodiments of the methods provided herein, a peptide is administered that has a target protein. A “target protein” is defined as a protein that the peptide modulated, changes, increases, decreases, or alters the activity, function, or binding partners of relative to cell or subject that did not receive treatment, or relative to a reference value or a threshold level. Target proteins of a peptide described herein (e.g., a peptide having a sequence of any one of SEQ ID NOs: 1-65) or of a recombinant host cell described herein can include a CCR9 protein, a CXCR3 protein, or a CXCR4 protein.

In some embodiments, the one or more target proteins is selected from the group consisting of a CCR9 protein, a CXCR3 protein, and a CXCR4 protein (e.g., as a human target for a peptide having a sequence of any one of SEQ ID NOs: 1-65). In some embodiments, the one or more target proteins is selected from the group consisting of a CCR9 protein, a CXCR3 protein, and a CXCR4 protein target for a peptide having a sequence of SEQ ID NO: 51).

In some embodiments, the CXCR3 and/or CCR9 protein is increased or upregulated during treatment with any of the peptides disclosed herein (e.g. peptides having the amino acid sequence of SEQ ID NO: 1, 46, or 51). In some embodiments, the CXCR3 and/or CCR9 target protein is not increased or upregulated during treatment with any of the peptides disclosed herein (e.g. peptides having the amino acid sequence of SEQ ID NO: 1, 46, or 51).

In some embodiments, the CXCR4 protein is decreased or downregulated during treatment with any of the peptides disclosed herein (e.g. peptides having the amino acid sequence of SEQ ID NO: 1, 46, or 51). In some embodiments, the CXCR4 protein is not decreased or downregulated during treatment with any of the peptides disclosed herein (e.g. peptides having the amino acid sequence of SEQ ID NO: 1, 46, or 51).

Suitable reference values can be determined using methods known in the art, e.g., using standard clinical trial methodology and statistical analysis. The reference values can have any relevant form. In some cases, the reference comprises a predetermined value for a meaningful level of one or more target proteins (e.g. a CCR9 protein, a CXCR3 protein, or a CXCR4 protein), e.g., a control reference level that represents a normal level of one or more target proteins e.g., a level in an unaffected subject or a subject who is not at risk of developing a disease described herein, and/or a disease reference that represents a level of the proteins associated with conditions associated with cancer, e.g., a level in a subject having any of the cancers described herein.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g. any of the cancers described herein). In some cases it may be desirable that the control subject has cancer, and in other cases it may be desirable that a control subject does not have cancer.

### Cancer

Globally suppressed T cell function has been described in many subjects with cancer to be a major hurdle for the development of clinically efficient cancer immunotherapies. The inhibition of antitumor immune responses has largely been linked to inhibitory factors present in subjects presenting with cancer. A “neoplastic disorder” is any disorder associated with cell proliferation, specifically with a neoplasm. A “neoplasm” or “neoplasia” is an abnormal mass of tissue that may be benign or malignant. Nearly all benign tumors are encapsulated and are non-invasive. In contrast, malignant tumors are almost never encapsulated and invade adjacent tissue by infiltrative destructive growth. This infiltrative growth can be followed by tumor cells implanting at sites discontinuous with the original tumor.

A neoplasm or a neoplastic disorder can be a cancer. “Cancer” as used herein refers to an uncontrolled growth of cells which interfere with the normal functioning of the bodily organs and systems. Hemopoietic cancers, such as leukemia, are able to outcompete the normal hemopoietic compartments in a subject, thereby leading to hemopoietic failure in the form of anemia, thrombocytopenia and neutropenia; ultimately causing death.

Cancers which migrate from their original location and seed vital organs can eventually lead to the death of the subject through the functional deterioration of the affected organ(s). A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function assays, chest X-rays and bone scan, in addition to the monitoring of specific symptoms.

Methods of the present disclosure may be utilized to treat or prevent neoplastic disorders in humans, including but not limited to cancers such as sarcoma, carcinoma, fibroma, leukemia, lymphoma, melanoma, myeloma, neuroblastoma, rhabdomyosarcoma, retinoblastoma, and glioma. Cancers include but are not limited to basal cell carcinoma, biliary tract cancer, bladder cancer, bone cancer, brain and central nervous system (CNS) cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, lung cancer (small cell and non-small cell), lymphoma (including Hodgkin's and non-Hodgkin's), melanoma, myeloma, neuroblastoma, oral cavity cancer (lip, tongue, mouth, and pharynx), ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, as well as other carcinomas and sarcomas.

In some embodiments of the method described herein, cancers can include melanoma, lung cancer, kidney cancer, bladder cancer, a head and neck cancer, Merkel cell carcinoma, urothelial cancer, breast cancer, glioblastoma, gastric cancer, a nasopharyngeal neoplasm, colorectal cancer, hepatocellular carcinoma, ovarian cancer, and/or pancreatic cancer.

In some embodiments, the subject has a hematological malignancy. Hematological malignancies can include multiple myeloma, non-Hodgkin lymphoma, Hodgkin lymphoma, diffuse large B-cell lymphoma, and chronic lymphocytic leukemia/small lymphocytic lymphoma (CLL/SLL).

“A subject having cancer” or “a subject identified as having cancer” is a subject that has been diagnosed with a cancer. In some embodiments, the subject has a cancer type characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture, or weight of the tissue.

Some primary tumors are not palpable and can be detected only through medical imaging techniques such as X-rays or by needle aspirations. The use of these latter techniques is more common in early detection. Molecular and phenotypic analysis of cancer cells within a tissue will usually confirm if the cancer is endogenous to the tissue or if the lesion is due to metastasis from another site.

It has been estimated that almost half of all currently diagnosed cancers will be treated with some form of cancer medicament. However, many forms of cancer, including melanoma, colorectal, prostate, endometrial, cervical, and bladder cancer do not respond well to treatment with cancer medicaments. In fact, only about 5-10 percent of cancers can be cured using cancer medicaments alone. These include some forms of leukemias and lymphomas, testicular cancer, choriocarcinoma, Wilm's tumor, Ewing's sarcoma, neuroblastoma, small-cell lung cancer, and ovarian cancer. Treatment of still other cancers, including breast cancer, requires a combination of therapy of surgery or radiotherapy in conjunction with a cancer medicament. See Bratzler and Peterson.

The tumor environment is often refractory to immunological attack. It is desirable in cancer immunotherapy to make the tumor environment less refractory so as to increase the activity of CTLs or other effector T cells within the tumor and to improve the overall efficacy of treatment. As used herein, “efficacy” refers to the ability of a chemotherapeutic and/or immunological composition or a combination treatment thereof to achieve a desired action or result.

It has been demonstrated that some human cancer patients develop an antibody and/or T lymphocyte response to antigens on neoplastic cells. It has also been shown in animal models of neoplasia that enhancement of the immune response can result in rejection or regression of that particular neoplasm. Molecules that enhance the T lymphocyte response in the mixed lymphocyte reaction (MLR) have utility in vivo in enhancing the immune response against neoplasia. Molecules which enhance the T lymphocyte proliferative response in the MLR (or small molecule agonists or antibodies that affected the same receptor in an agonistic fashion) can be used therapeutically to treat cancer. Molecules that inhibit the lymphocyte response in the MLR also function in vivo during neoplasia to suppress the immune response to a neoplasm; such molecules can either be expressed by the neoplastic cells themselves or their expression can be induced by the neoplasm in other cells. Antagonism of such inhibitory molecules (either with antibody, small molecule antagonists or other means) enhances immune-mediated tumor rejection.

### Clinical Parameters for Treating Neoplasia

The administration of a composition comprising a peptide of the present disclosure results in a biological response in the subject/subject's cells. In some embodiments, administration of one or more peptides of the present disclosure results in the subject or the cells isolated therefrom to exhibit one or more of a reduction in the expression of IL-10, an increase of inflammatory (pro-inflammatory) cytokines, an increase of TNF-α, a reduction in anti-inflammatory cytokines, a limiting of tolerogenic dendritic cell expansion, a reduction in the ratio of IL-10:TNF, increase in the expression of IL-12, an increase or promotion of Th1 activation, an increase in TNF, an increase or enhancement of dendritic cell maturation, an increase in CD70 expression, an increase in T-cell activation, an increase in T-cell activation along with co-stimulation via CD27, an increase in the expression of CD80 and/or CD86, an increase or the enhancement of T-cell activation, an increase in T-cell activation along with co-stimulation via CD28, an increase in the expression of MHC I and/or MHC II, an increase or enhancement of T-cell activation by means of an increase in MHC-involved antigen presentation, a decrease in the number of T_{reg} cells, preventing the clonal expansion of T_{reg} cells and/or promoting the clonal expansion of T_{eff} cells, an increase in the number of Tact cells, an increase in the number of CTL cells, a decrease in the size and/or volume of neoplastic tissue, preventing metastasis of neoplastic tissue or cells, induction of apoptosis in neoplastic cells, an increase in the rate of apoptosis of neoplastic cells, a reduction in the number of neoplastic masses in one or more tissues, a decrease in the size of neoplastic lesions, and an increase in the clonal expansion of T_{act}, T_{eff/mem}, and/or CTL cells.

In some embodiments, administration of a composition comprising the peptide to a subject results in an increased life expectancy in the subject of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some embodiments, administration of a composition comprising the peptide to a subject results in an increased life expectancy in the subject of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In some embodiments, administration of a composition comprising the peptide to a subject results in an increased life expectancy in the subject of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years.

In some embodiments, administration to a subject of a composition comprising the peptide results in a reduction in the volume of one or more neoplasia by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 7⁶%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 98% of the volume of the one or more neoplasia.

In some embodiments, administration to a subject of a composition comprising the peptide results in a reduction of the size of one or more neoplastic lesions by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, or 98% of the volume of the one or more neoplastic lesions.

In some embodiments, administration to a subject of a composition comprising the peptide results in a reduction in one or more negative side effects of the neoplasia by at least 5%, 10%, 20%, 25%, 30%, 35%, 40%, 45%, 50%, 55%, 60%, 65%, 70%, 75%, 80%, 85%, 90%, or 95%; wherein the negative side effects include nausea, pain, discomfort, vomiting, diarrhea, vertigo, loss of appetite, nerve pain, seizures, periodic loss of consciousness, loss or lack of ambulatory movement, loss or lack of physical coordination, and loss or lack of vision.

In some embodiments, administration to a subject of a composition comprising the peptide results in a shift in the clonal populations of T_{reg} and T_{eff} cells in contact with the one or more neoplasia, wherein the population of T_{eff} increases at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30-fold relative to the population of T_{reg}.

### Combination Therapies Comprising Peptides

The pharmaceutical compositions provided herein comprising a peptide may be combined with additional/other treatment therapies (e.g., treatment(s) for cancer) and/or pharmaceutical compositions. For example, a subject suffering from an immunological associated disease or disorder, or cancer, may already be taking a pharmaceutical prescribed by their doctor to treat the condition. In embodiments, the pharmaceutical compositions provided herein, are able to be administered in conjunction with the subject's existing medicines.

For example, the peptides provided herein may be combined with one or more of: a 5-aminosalicylic acid compound, an anti-inflammatory agent, an antibiotic, an antibody (e.g. antibodies targeting an inflammatory cytokine, e.g. antibodies targeting TNF-α, such as adalimumab, pegol, golimumab, infliximab, and certolizumab), an anti-cytokine agent, an anti-inflammatory cytokine agent, a steroid, a corticosteroid, an immunosuppressant (e.g. azathioprine and mercaptopurine), vitamins, and/or specialized diet. In some embodiments, the peptide of the present disclosure is administered with a checkpoint inhibitor, such as an agent that targets PD-1, PD-L1, CTLA-4, BTLA, LAG-3, A2AR, TIM-3, B7-H3, VISTA, or IDO. Such drugs include but are not limited to pembrolizumab, nivolumab, atezolizumab, avelumab, durvalumab, cemiplimab, and ipilimumab. The peptides provided herein may be combined with an autologous cellular immunotherapy (e.g., sipuleucel-T). In some embodiments, the other treatment therapies and/or pharmaceutical compositions may be selected from cancer immunotherapies such as monoclonal antibodies that activate NK cells and enhance antibody-dependent cellular cytotoxicity; cancer vaccines with or without adjuvants that stimulate a cancer-antigen-specific humoral immune response; chemotherapeutic agents such as carboplatin and/or mitotane; hormones such as adrenocorticosteroids or fluoxymesterone; or biological response modifiers that alter a subject's response to cancer rather than by direct cytotoxicity of cancer cells, such as erythropoietin or GM-CSF. An extensive, but non-limiting list of treatment therapies, pharmaceutical compositions/medicaments are disclosed in Bratzler and Peterson.

In some embodiments, one or more tumors or neoplastic tissues are debulked prior to or during immunotherapy. In some embodiments, debulking one or more tumors prior to or during immunotherapy results in either a slowing or a halt of disease progression. In some embodiments, debulking one or more tumors prior to or during immunotherapy results in tumor regression or elimination.

In some embodiments, a slowing of disease progression comprises at least a 1%, 2%, 3%, 4%, 5%, 6%, 7%, 8%, 9%, %, %11%, %12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% decrease in the rate of growth or expansion of the tumor.

In some embodiments, tumor regression comprises at least a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%6, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% decrease in the tumor numbers, tumor size, or tumor volume.

Any procedure that allows an assessment of the tumor or lesion size can be used. Non-limiting examples include digital rectal exam, an endoscopy (e.g., a colonoscopy), and imaging (e.g., PET, MRI, ERUS, DRE, CT). See, for example, McKeown et al. J Cancer. 2014; 5(1): 31-43. In some embodiments, tumor burden can be assessed using RECIST (e.g., RECIST version 1 or version 1.1). See, for example, Eisenhauer et al., Eur. J. Cancer. 45(2):228-47 (2009).

Criteria for evaluating immunotherapy have also been developed. Non-limiting examples include the immune-related response criteria (irRC) (see Wolchok et al. Clin Cancer Res. 2009 Dec. 1; 15(23):7412-20) and immune response criteria in solid tumors (iRECIST) criteria (see Seymour et al. Lancet Oncol. 2017 March; 18(3): e143-e152). See also, Thallinger et al. Wien Klin Wochenschr. 2018; 130(3): 85-91.

Having an antigen-specific humoral and cell-mediated immune response, in addition to activating NK cells and endogenous dendritic cells, and increasing IFN levels, can be helpful for treating cancer. Some cancer cells are antigenic and thus can be targeted by the immune system. In one aspect, peptides of the present disclosure, or peptides of the present disclosure plus one or more cancer medicaments are particularly useful for stimulating an immune response against a cancer. A “cancer antigen” as used herein is a compound, such as a peptide, associated with a tumor or cancer cell surface and which is capable of provoking an immune response when expressed on the surface of an antigen presenting cell, such as a dendritic cell. In some aspects, the antigen is presented on an APC via an MHC molecule. Cancer antigens, such as those present in cancer vaccines or those used to prepare cancer immunotherapies, can be prepared from crude cancer cell extracts or by partially purifying the antigens, using recombinant technology or de novo synthesis of known antigens. In some aspects, proteins isolated from other organisms or synthetic proteins sharing a degree of homology to the proteins are used to prepare cancer immunotherapies.

Different types of cells that can kill tumor targets in vitro and in vivo have been identified: natural killer cells (NK cells), cytolytic T lymphocytes (CTLs), lymphokine-activated killer cells (LAKs), and activated macrophages. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tumors and of tumors caused by DNA viruses. LAK cells are a subset of null lymphocytes distinct from the NK and CTL populations. Activated macrophages can kill tumor cells in a manner that is not antigen dependent nor MHC restricted once activated. Activated macrophages are thought to decrease the growth rate of the tumors they infiltrate. In vitro assays have identified other immune mechanisms such as antibody-dependent, cell-mediated cytotoxic reactions and lysis by antibody plus complement. However, these immune effector mechanisms are thought to be less important in vivo than the function of NK, CTLs, LAK, and macrophages.

The use of peptides of the present disclosure in conjunction with cancer vaccines provides an improved antigen-specific humoral and cell-mediated immune response, in addition to activating NK cells and endogenous dendritic cells, and increasing IFN levels. Such an enhancement can allow for the use of a vaccine with a reduced antigen dose to achieve the same beneficial effect.

### Pharmaceutical Compositions Comprising Peptides

Compositions are provided that comprise a peptide as described herein. In some embodiments, the compositions described herein are pharmaceutical compositions suitable for administration to as subject and that demonstrate a therapeutic effect when administered to a subject in need thereof. Pharmaceutical compositions of the present disclosure can comprise a therapeutically effective amount of a peptide in a pharmaceutically acceptable carrier. The preparation of a pharmaceutical composition or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by the FDA Office of Biological Standards.

The compositions of the disclosure can comprise different types of carriers depending on whether it is to be administered in solid, liquid or aerosol form, and whether it needs to be sterile for such routes of administration as injection. The peptides of the disclosure can be administered orally, or rectally, but can also be administered intrathecally, intranasally, subcutaneously, mucosally, by inhalation (e.g., aerosol inhalation), by injection, by infusion or continuous infusion, topically, localized perfusion bathing target cells directly, via a catheter, via a lavage, or by other method or any combination of the foregoing as would be known to one of ordinary skill in the art (see, for example, Remington's Pharmaceutical Sciences, 18^{th} Ed. Mack Printing Company, 1990, incorporated herein by reference).

The peptides of the present disclosure can be formulated into a composition in a free base, neutral, or salt form. Pharmaceutically acceptable salts, include the acid addition salts, e.g., those formed with the free amino groups of a proteinaceous composition, or which are formed with inorganic acids such as, for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium, or ferric hydroxides; or such organic bases as isopropylamine, trimethylamine, histidine, or procaine.

In some embodiments where the composition is in a liquid form, a carrier can be a solvent or dispersion medium comprising but not limited to water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In particular embodiments, the peptide compositions of the present disclosure are prepared for administration by such routes as oral ingestion (e.g. oral administration). In these embodiments, the solid composition can comprise, for example, solutions, suspensions, emulsions, tablets, pills, capsules (e.g., hard or soft shelled gelatin capsules), delayed release capsules, sustained release formulations, buccal compositions, troches, elixirs, suspensions, syrups, wafers, or combinations thereof. Oral compositions can be incorporated directly with the food of the diet. Preferred carriers for oral administration can comprise inert diluents, assimilable edible carriers, or combinations thereof. In other aspects of the disclosure, the oral composition can be prepared as a syrup or elixir. A syrup or elixir, and can comprise, for example, at least one active agent, a sweetening agent, a preservative, a flavoring agent, a dye, a preservative, or combinations thereof.

In some embodiments, an oral composition can comprise one or more binders, excipients, disintegration agents, lubricants, flavoring agents, and combinations thereof. In some embodiments, a composition can comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it can contain, in addition to materials of the above type, carriers such as a liquid carrier. Various other materials can be present as coatings or to otherwise modify the physical form of the dosage unit. For instance, tablets, pills, or capsules can be coated with shellac, sugar or both.

Additional formulations which are suitable for other modes of administration include suppositories (e.g. rectal administration). Suppositories are solid dosage forms of various weights and shapes, usually medicated, for insertion into the rectum. After insertion, suppositories soften, melt or dissolve in the cavity. In general, for suppositories, traditional carriers can include, for example, polyalkylene glycols, triglycerides or combinations thereof. In some embodiments, suppositories can be formed from mixtures containing, for example, the active ingredient in the range of about 0.5% to about 10%, and preferably about 1% to about 2%.

In some embodiments, compositions suitable of intravenous administration are provided. In some embodiments, compositions suitable for intratumoral administration are provided. In some embodiments, compositions suitable for parenteral administration are provided. Injectable formulations comprise one or more described peptides in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain sugars, alcohols, amino acids, antioxidants, buffers, bacteriostats, solutes which render the formulation isotonic with the blood of the intended recipient or suspending or thickening agents. Examples of suitable aqueous and nonaqueous carriers which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate.

These pharmaceutical compositions can also contain adjuvants such as preservatives, wetting agents, emulsifying agents, and dispersing agents. Prevention of the action of microorganisms upon the described compounds can be ensured by the inclusion of various antibacterial and antifungal agents, for example, paraben, chlorobutanol, phenol sorbic acid, and the like. It can also be desirable to include agents to control tonicity, such as sugars, sodium chloride, and the like into the compositions. In addition, prolonged absorption of the injectable pharmaceutical form can be brought about by the inclusion of agents which delay absorption such as aluminum monostearate and gelatin.

Sterile injectable solutions can be prepared by incorporating the active compounds, e.g., a peptide described herein, in the required amount in the appropriate solvent with various combinations of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of a peptide described herein plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein/peptide.

In some embodiments, the composition comprises a purified peptide that comprises, consists of, or consists essentially of, any of the amino acid sequences listed in Table 1, 2, or 3 (e.g., peptides having SEQ ID Nos 1-51).

### Production or Synthesis of Peptides

Any of the peptides described herein can be chemically synthesized. Method of peptide chemical synthesis are known in the art. See, for example, Isidro-Llobet et.al. 2019. Journal of Organic Chemistry.84, 8, 4615-4628 or Stawikowski and Fields. 2002. Curr. Protoc. Protein Sci. doi: 10.1002/0471140864.ps1801s26, for methods of chemical synthesis of peptides.

Any of the peptides described herein can be generated using recombinant techniques. For example, a polynucleotide sequence encoding the peptide, e.g., any of the peptides described herein, or a variant thereof, can be cloned into a nucleic acid construct (e.g., an expression vector), which can be used to transform an appropriate host cell, e.g., a prokaryote (e.g., a eubacteria, an archaea) or a eukaryote (yeast). In some embodiments, the host cells can be, e.g., E. coli BL21 cells, Bacillus sp. such as B. thuringiensis, or P. fluorescens.

Numerous cell lines and cultures are available for use as a host cell, and they can be obtained for example through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5u, JM109, and KC8, bacilli such as Bacillus subtilis and Bacillus thuringiensis; and other enterobacteriaceae such as Salmonella typhimurium, Serratia marcescens, various Pseudomonas species such as P. fluorescens, as well as a number of commercially available bacterial hosts such as SURE® Competent Cells and SOLOPACK™ Gold Cells (STRATAGENE®, La Jolla). In some embodiments, bacterial cells such as E. coli are particularly contemplated as host cells.

Examples of eukaryotic host cells for replication and/or expression of a vector include, but are not limited to, HeLa, NIH3T3, Jurkat, 293, Cos, Chinese Hamster Ovary (CHO), Saos, and PC12. Many host cells from various cell types and organisms are available and would be known to one of skill in the art. Similarly, a viral vector can be used in conjunction with either a eukaryotic or prokaryotic host cell, particularly one that is permissive for replication or expression of the vector.

From a given amino acid sequence (e.g. any of the sequences in Tables 1-7), the nucleic acid sequence can be codon optimized (e.g., using a codon optimization algorithm) to generate the nucleotide sequence. The codon optimization algorithm chooses an appropriate codon for a given amino acid based on the expression host's codon usage bias. Many codon optimization algorithms also take into account other factors such as mRNA structure, host GC content, ribosomal entry sites. Some examples of codon optimization algorithms and gene synthesis service providers are: GenScript: genscript.com/codon-opt.html on the World Wide Web; ThermoFisher: thermofisher.com/us/en/home/life-science/cloning/gene-synthesis/geneart-gene-synthesis/geneoptimizer.html on the World Wide Web; and Integrated DNA Technologies: idtdna.com/CodonOpt on the World Wide Web. The nucleotide sequence is then synthesized and cloned into an appropriate nucleic acid construct (e.g., an appropriate expression vector).

The host cells containing the nucleic acid construct can be cultured to allow growth of the cells and expression of the peptide. In some embodiments, expressed peptide can then be purified, again using a variety of methods readily known to a person having ordinary skill in the art. Generally, “purified” will refer to a specific peptide composition that has been subjected to fractionation to remove non-proteinaceous components and various other proteins, polypeptides, or peptides, and which composition substantially retains its activity, as can be assessed, for example, by the protein assays, as described herein below, or as would be known to one of ordinary skill in the art for the desired protein, peptide or peptide.

Where the term “substantially purified” is used, this will refer to a composition in which the specific protein, peptide, or peptide forms the major component of the composition, such as constituting about 50% of the peptides in the composition or more. In preferred embodiments, a substantially purified peptide will constitute more than 60%, 70%, 80%, 90%, 95%, 99% or even more of the peptides in the composition. Purity can be measured with percent weight by weight or percent weight by volume, or any other customary purity measurement.

A peptide, polypeptide or protein that is “purified to homogeneity,” as applied to the present disclosure, means that the peptide, polypeptide or protein has a level of purity where the peptide, polypeptide or protein is substantially purified or free from other proteins/peptides and biological components. For example, a purified peptide, polypeptide or protein will often be sufficiently free of other protein/peptide components so that degradative sequencing can be performed successfully.

Various methods for quantifying the degree of purification of proteins, peptides, or peptides will be known to those of skill in the art in light of the present disclosure. These include, for example, determining the specific polypeptide activity of a fraction, or assessing the number of peptides within a fraction by gel electrophoresis.

Although preferred for use in some embodiments, there is no general requirement that the protein, polypeptide, or peptide always be provided in their most purified state. Indeed, it is contemplated that less substantially purified protein, polypeptide or peptide, which are nonetheless enriched in the desired peptide compositions, relative to the natural state, will have utility in some embodiments. As used herein “enriched” refers to a component, such as a peptide or protein, that is found at a high concentration in a solution or composition than in nature when produced the same way. Methods of enrichment of a component, such as enrichment of a peptide, are known in the art and are often similar to those of purification of components or peptides.

Methods exhibiting a lower degree of relative purification can have advantages in total recovery of peptide product, or in maintaining the activity of an expressed peptide. Inactive products also have utility in some embodiments, such as, e.g., in determining antigenicity via antibody generation.

In other embodiments, a preparation enriched with the peptides can be used instead of a purified preparation. In this document, whenever purified is used, enriched can be used also. A preparation can be enriched not only by methods of purification, but also by the over-expression or over-production of the peptide by bacteria when compared to wild-type. This can be accomplished using recombinant methods, or by selecting conditions which will induce the expression of the peptide from the wild type cells.

### Expression Systems

Provided herein are compositions and methods for producing peptides of the present disclosure. Also provided are nucleic acid constructs that contain a polynucleotide sequence encoding a peptide described herein. Also provided herein are host cells which harbor the nucleic acid constructs. The peptides of the present disclosure can be prepared by routine recombinant methods, e.g., culturing cells transformed or transfected with a nucleic acid construct (e.g., an expression vector) containing a nucleic acid encoding a peptide described herein. Numerous expression systems can be used to produce a peptide as discussed above. Prokaryote- and/or eukaryote-based systems can be employed for use with the present disclosure to produce nucleic acid sequences, or their cognate peptides, polyproteins and peptides. Many such systems are commercially and widely available. Expression systems include but are not limited to insect cell/baculovirus systems and inducible mammalian expression systems, it is contemplated that the proteins, polypeptides or peptides produced by the methods of the disclosure can be “overexpressed.” For example, proteins, peptides or peptides can be expressed in increased levels relative to its natural expression in cells.

Accordingly, a method for producing any of the herein described peptides is further provided and comprises culturing host cells under conditions suitable for expression of the desired peptide and recovering the desired peptide from the cell culture. The recovered peptide can then be isolated and/or purified for use in in vitro and in vivo methods, as well as for formulation into a pharmaceutically acceptable composition. In some embodiments, the peptide is expressed in a prokaryotic cell such as E. coli, Lactococcus lactis, Streptomyces species (e.g., S. coelicolor, S. lividans, S. albus, or S. venezuelae), or Bacillus species (e.g., B. subtilis). In some embodiments, the peptide is expressed in a eukaryotic cell such as a yeast (e.g., Saccharomyces cerevisiae, Pichia pastoris, Yarrowia lipolytica, Aspergillus niger, Hansenula polymorpha) or an insect cell (e.g., sf9, sf21, Tni, and S2). In some embodiments, the isolation and purification of the peptide includes one or more steps to reduce endotoxin to levels acceptable for therapeutic use in humans or other animals.

Also provided herein are nucleic acid constructs which comprise a polynucleotide sequence which encodes a peptide of the present disclosure. Polynucleotide sequences encoding the peptides of the disclosure can be obtained using standard recombinant techniques. Desired encoding polynucleotide sequences can be amplified from the genomic DNA of the source bacterium, e.g., Bacillus thuringiensis. Alternatively, polynucleotides can be synthesized using nucleotide synthesizer. Once obtained, sequences encoding the peptides are inserted into a recombinant vector capable of replicating and expressing heterologous (exogenous) polynucleotides in a host cell. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using a pBR322, pUC, pET or pGEX vector, a plasmid derived from an E. coli species. Such vectors can contain genes encoding ampicillin (Amp) and tetracycline (Tet) resistance and thus provides easy means for identifying transformed cells. These vectors as well as their variants or other microbial plasmids or bacteriophage can also contain, or be modified to contain, promoters which can be used by the microbial organism for expression of endogenous proteins.

A nucleic acid construct of the present disclosure can comprise a promoter, an untranslated regulatory sequence located upstream (5′) and operably linked to a polypeptide-encoding nucleotide sequence such that the promoter regulated transcription of that coding sequence. Prokaryotic promoters typically fall into two classes, inducible and constitutive. An inducible promoter is a promoter that initiates increased levels of transcription of the encoding polynucleotide under its control in response to changes in the culture condition, e.g., the presence or absence of a nutrient or a change in temperature. A large number of promoters recognized by a variety of potential host cells are well known and a skilled artisan can choose the promoter according to desired expression levels. Promoters suitable for use with prokaryotic hosts include E. coli promoters such as lac, trp, tac, trc and ara, viral promoters recognized by E. coli such as lambda and T5 promoters, and the T7 and T7lac promoters derived from T7 bacteriophage. A host cell harboring a vector comprising a T7 promoter, e.g., is engineered to express a T7 polymerase. Such host cells include E. coli BL21(DE3), Lemo2l(DE3), and NiCo2l(DE3) cells. Promoters suitable for use with yeast hosts include promoters such as yeast alcohol dehydrogenase 1 (ADH1) promoter, yeast phosphoglycerate kinase (PGK1) promoter, and translational elongation factor EF-1 alpha promoter. In some embodiments, wherein the host cell is a H. polymorpha cell, the promoter is a MOX promoter. In some embodiments, the promoter is an inducible promoter which is under the control of chemical or environmental factors.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Suitable vectors for expression in both prokaryotic and eukaryotic host cells are known in the art.

Vectors of the present disclosure can further comprise a signal sequence which allows the translated recombinant peptide to be recognized and processed (i.e., cleaved by a signal peptidase) by the host cell. For prokaryotic host cells that do not recognize and process the signal sequences native to the heterologous peptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PeIB, OmpA and MBP. Well-known signal sequences for use in eukaryotic expression systems include but are not limited to interleukin-2, CD5, the Immunoglobulin Kappa light chain, trypsinogen, serum albumin, and prolactin.

The peptides as described herein (e.g., a peptide comprising a sequence from Tables 1-7) can be expressed as a fusion protein or peptide. Commonly used fusion partners include but are not limited to human serum albumin and the crystallizable fragment, or constant domain of IgG, Fc. The histidine tag or FLAG tag can also be used to simplify purification of recombinant peptide from the expression media or recombinant cell lysate. The fusion partners can be fused to the N- and/or C-terminus of the peptide of interest.

Methods are well known for introducing recombinant DNA, i.e., a nucleic acid construct such as an expression vector, into a host cell so that the DNA is replicable, either as an extrachromosomal element or as a chromosomal integrant, thereby generating a host cell which harbors the nucleic acid construct of interest. Methods of transfection are known to the ordinarily skilled artisan, for example, by CaPO4 and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. General aspects of mammalian cell host system transformations have been described in U.S. Pat. No. 4,399,216. Transformations into yeast are can be carried out according to the method of Van Solingen et al., J Bact, 130:946 (1977) and Hsiao et al., Proc. Natl. Acad. Sci. (USA), 76:3829 (1979). Other methods for introducing DNA into cells include nuclear microinjection, electroporation, bacterial protoplast fusion with intact cells, or introduction using polycations, e.g., polybrene, polyornithine. For various techniques for transforming mammalian cells, see Keown et al., Methods in Enzymology. 185:527-537 (1990) and Mansour et al., Nature, 336:348-352 (1988).

Accordingly, provided herein is a nucleic acid construct (e.g., a recombinant vector or expression vector) as described above and comprising a polynucleotide which encodes a peptide sequence of interest (e.g., any of the peptide described herein, such as the peptide of SEQ ID NO: 1, 46, or 51). Moreover, the present disclosure provides a host cell harboring the vector. The host cell can be a eukaryotic or prokaryotic cell as detailed above. In some embodiments, the host cell is a prokaryotic cell. In some embodiments, the host cell is E. coli, L. lactis, S. coelicolor, S. lividans, S. albus, S. venezuelae, or B. subtilis.

### EXAMPLES

The invention is further described in the following examples, which do not limit the scope of the invention described in the claims.

### Example 1. Identifying Peptides that Induce CXCL10 Production

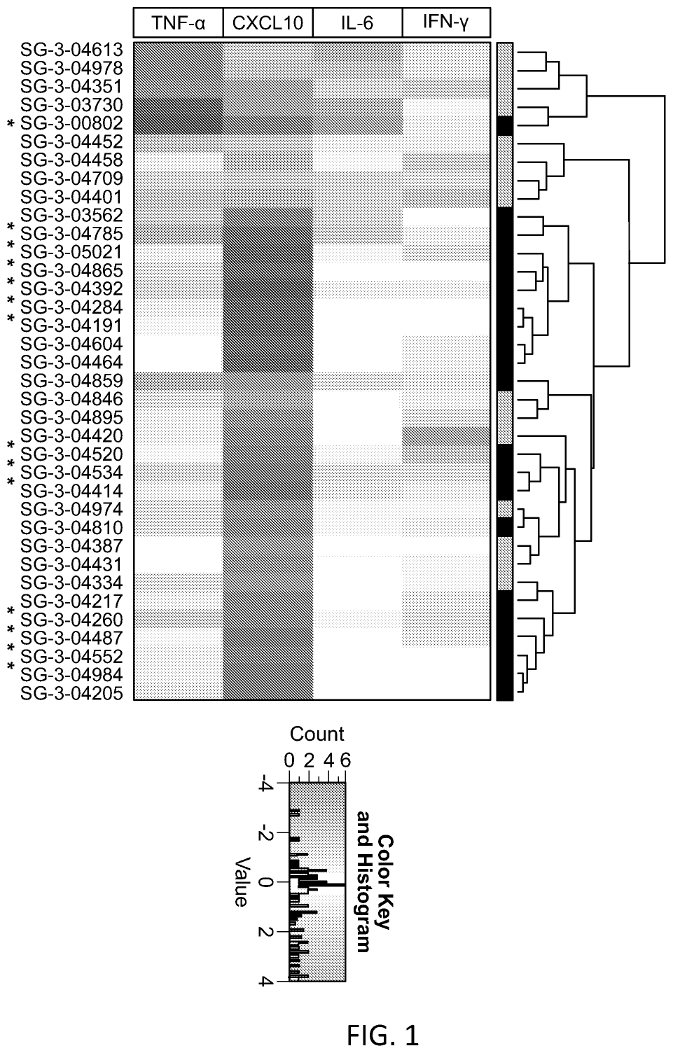

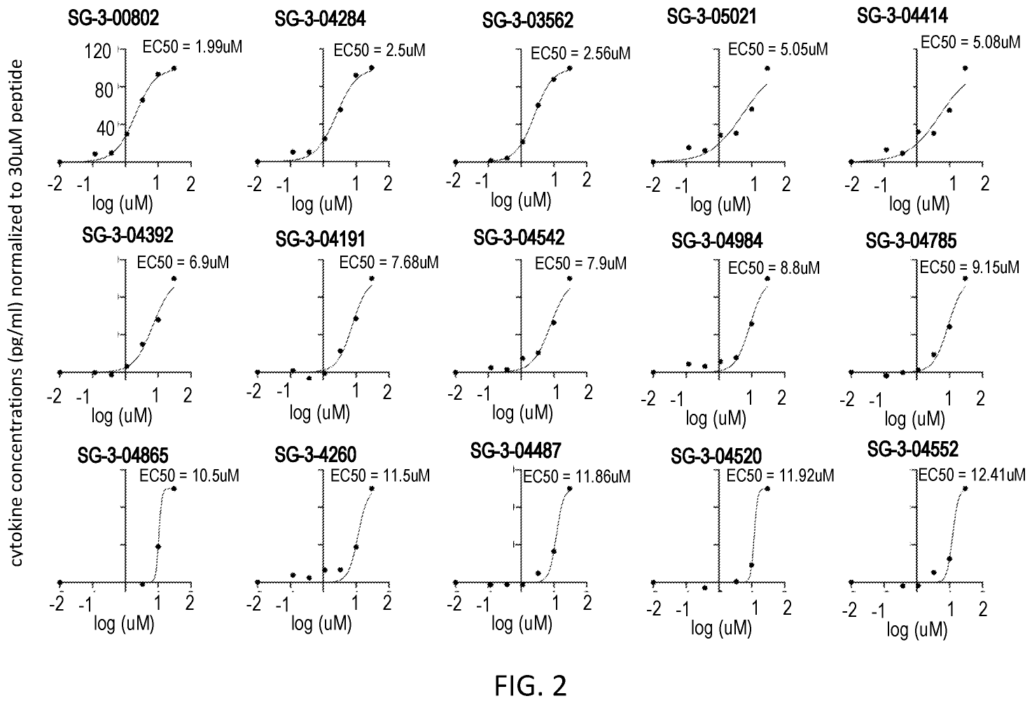

Studies were performed to identify target peptides that induced CXCL10 production. Target peptides identified from human microbiomes from patients that responded to anti-PD-1 therapy were each synthesized and tested for TNF-α, CXCL10, IL6, and INF-γ induction. Fifteen peptides with high CXCL10 induction were identified for additional testing, indicated with asterisks in the figure (FIG. 1). CXCL10 was up-regulated for all peptides indicated with an asterisk compared to other peptides and a negative control peptide (chicken ovalbumin peptide 323-339) (FIG. 1). Peptides SG-3-00802 and SG-3-05021 were identified as high CXCL10 inducers (FIG. 2). Peptide sequences and characteristics can be found in Tables 4-7.

TABLE 4
Peptide Sequence and Strains

| | Peptide | | |
|---|---|---|---|
| Peptide ID | name | Sequence | Strain or RDM |
| SEQ ID NO: 1 | SG-3-00802 | MKVRPSVKPMCEKCKIIRRKGRVMVICENPK | oscillibacter.sp |
| | | HKQRQG | t_155102 |
| | | | (GCF_000765235) |
| SEQ ID NO: 51 | SG-3-05021 | LGKTDWIEKYFKVKKEKIDKMQRFLQG | bacteroides.clarus.dsm. |
| | | | 22519 |
| | | | t_58760 |
| | | | (GCF_000195615) |

TABLE 5
Peptide Characteristics

| | Full Length vs | Contained/overlap | PFAM |
|---|---|---|---|
| Peptide ID | fragment? | with a PFAM region? | notes |
| SEQ ID | Full | | |
| NO: 1 | | | |
| SEQ ID | TMHMM-o | Contained within PFAM region. Source protein has 2 | DUF2238 relates to |
| NO: 51 | | PFAM regions (DUF2238 and SNARE_assoc) from | an inner membrane |
| | | positions 51-136 and 35-147. The TMHMM-o | protein |
| | | fragment here is from 73-100 | |

TABLE 6
Peptide Characteristics.

| | Secreted: | Secreted: | Secreted: | | | |
|---|---|---|---|---|---|---|
| Peptide ID | SigP | SecP | PsortB | pI | Length (aa) | Binder? |
| SEQ ID NO: 1 | No | No prediction | No | 10.69469949 | 37 | DC |
| SEQ ID NO: 51 | No | Yes | No | 9.777983413 | 27 | DC |

TABLE 7
Additional Peptide Sequences.

| SEQ ID NO: | Peptide Name | Sequence |
|---|---|---|
| 53 | SG-3-04865 | GDTIVEWYRTARARQTFLHKRTWQKNLRAFV |
| 54 | SG-3-04534 | MWSVVKKIIALSGDKNKYIK |
| 55 | SG-3-04552 | IRKKVDEKKKQLQKVQTSFHQKVSSI |
| 56 | SG-3-04785 | LYQQYIHPIVMSLLRKKSMTKPRVNTGVL |
| 57 | SG-3-04520 | MGPLIQWFQKNWIKKVIEK |
| 58 | SG-3-04284 | TKKIPWSFWKKSKNPAVR |
| 59 | SG-3-04191 | KNRIEKWWPCKKYLVAHRR |
| 60 | SG-3-04414 | MKEKIKKMPGRIRNYFCS |
| 61 | SG-3-04260 | NMSGWREFVKLVRKSPKS |
| 62 | SG-3-04984 | FLALLGGNGTGKTTTLKLLANLKKAYR |
| 63 | SG-3-04487 | MLKKILYYGKENNKKTVR |
| 64 | SG-3-03562 | MKVRPSVKPMCDKCRVIKRKGRVMIICENPKHKQRQG |
| 65 | SG-3-04392 | MKNQSHPIVIVKKRKHKAHGHGSHGSW |

### Example 2. Modifying SG-3-00802 (SEQ ID NO: 1) for Increased Stability Generated SG-3-06683 (SEQ ID NO: 46)

Further modifications were performed to SG-3-00802 (MKVRPSVKPMCEKCKIIRRKGRVMVICENPKHKQRQG, SEQ ID NO: 1) to increase proteolytic stability. Modifications included converting the peptides to a retro-inverso peptide, which resulted in peptide SG-3-06683 (GQRQKHKPNEC IVMVRGKRRIIKCKECMPKVSPRVKM, SEQ ID NO: 46). By using D-amino acids and reversing the sequence of the peptide, the global folding was maintained, while only reversing the direction of the backbone units. Activation of CXCR3 by SG-3-06683 was maintained.

### Example 3. Identification of CCRs as Potential Targets for SG-3-00802 (SEQ ID NO: 1) by Mass Spectrometry

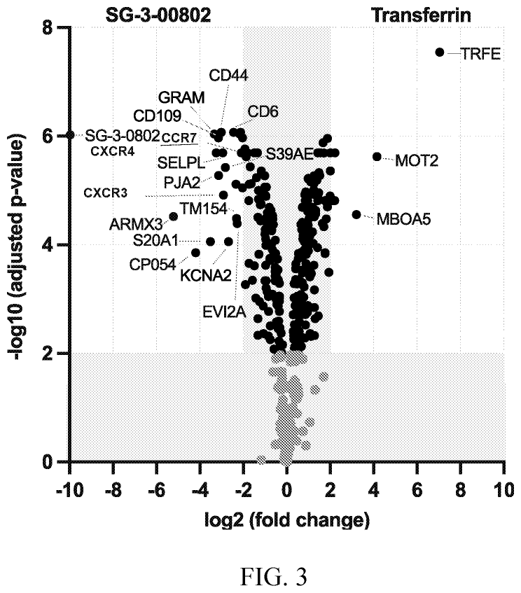

SG-3-00802 binding partners were identified using the mass spectrometry-based proteomics (LC MS/MS)-based LRC-TriCEPS™ technology from DualSystems. To enrich for binding partners the ligand was coupled with the LRC-TriCEPS™ crosslinker and incubated with living cells that expressed target receptors. The enriched proteins were purified and processed for analysis by LC-MS/MS. A parallel experiment using transferrin as control ligand enabled the relative quantification of the enriched proteins. Putative binding partners of SG-3-00802 enriched over transferrin ligand are depicted in a volcano plot (FIG. 3). Chemokine receptors were identified as the binding partners of SG-3-00802 (SEQ ID NO: 1).

### Example 4. Assaying Agonist, Antagonist, and Positive Allosteric Modulator Function of SG-3-00802 and SG-3-05021 Peptides

Peptides SG-3-00802 and SG-3-05021 were screened for agonist and antagonist activity of the chemokine receptors CCR7, CXCR3, and CXCR4.

In agonist assays, chemokine receptor expressing U2OS cells are thawed and resuspended in Assay Media (DMEM, 1% dialyzed FBS, 25 mM HEPES pH 7.3, 0.1 mM NEAA, 100 U/mL/100 μg/mL Pen/Strep) to a concentration of 312,500 cells/mL. 4 μL of a 10× serial dilution of I-TAC (control agonist starting concentration, 500 nM) or compounds are added to appropriate wells of a 384-well TC-Treated assay plate. 32 μL of cell suspension (10,000 cells) is added to each well. 4 μL of Assay Media is added to all wells to bring the final assay volume to 40 μL. The plate is incubated for 16-24 hours at 37° C./5% CO₂ in a humidified incubator. 8 μL of 1 μM Substrate+Solution D Loading Solution is added to each well and the plate is incubated for 2 hours at room temperature. The plate is read on a fluorescence plate reader.

In antagonist assays, CXCR3-bla U2OS cells were thawed and prepared as described above for the Agonist assay. 4 μL of 10× compounds or assay media was added to appropriate wells of a TC-Treated assay plate. 32 μL of cell suspension was added to the wells and pre-incubated at 37° C./5% CO₂ in a humidified incubator with compounds and control antagonist titration for 30 minutes. 4 μL of 10× control agonist I-TAC at the pre-determined EC80 concentration is added to wells containing the control antagonist or compounds. The plate is incubated for 16-24 hours at 37° C./5% CO₂ in a humidified incubator. 8 μL of 1 μM Substrate+Solution D Loading Solution is added to each well and the plate is incubated for 2 hours at room temperature. The plate is read on a fluorescence plate reader. At this time, the CXCR3-bla U2OS assay does not have an antagonist control.

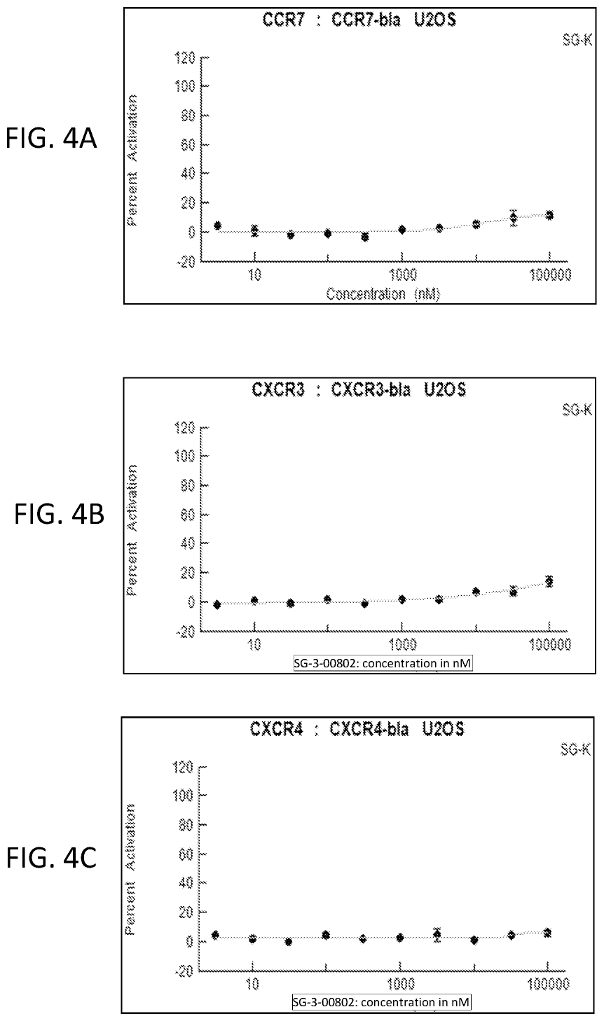

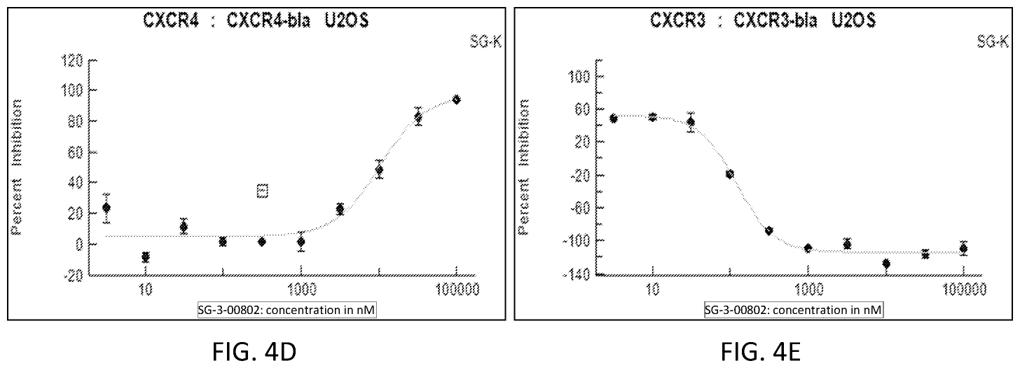

SG-3-00802 showed weak agonistic activity for CCR7 (FIG. 4A) and CXCR3 (FIG. 4B), no agonistic activity for CXCR4 (FIG. 4C), a moderate antagonistic activity for CXCR4 (FIG. 4D). SG-3-00802 shows potent reverse antagonistic activity (sensitization) for CXCR3 (FIG. 4E).

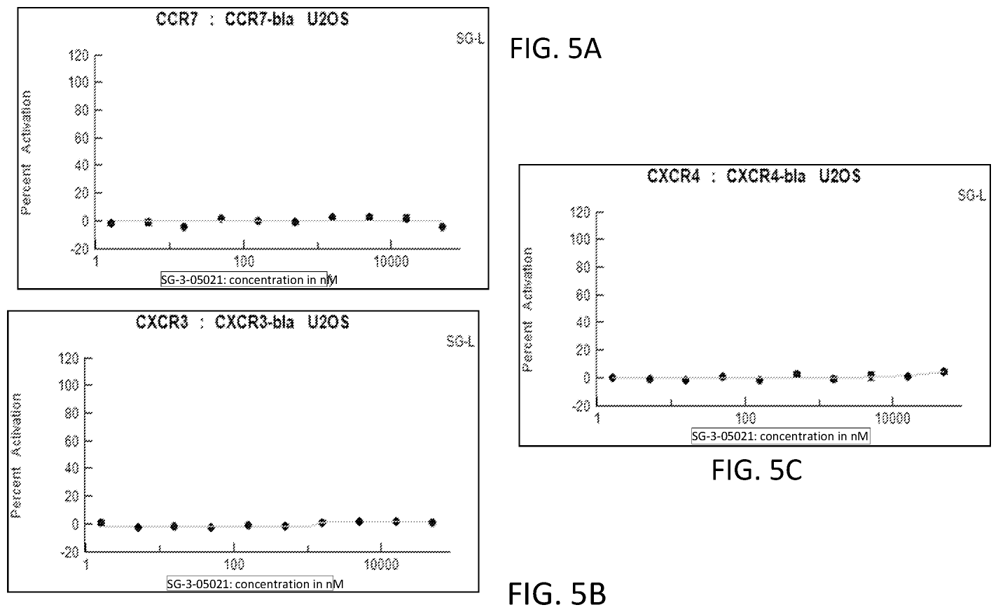

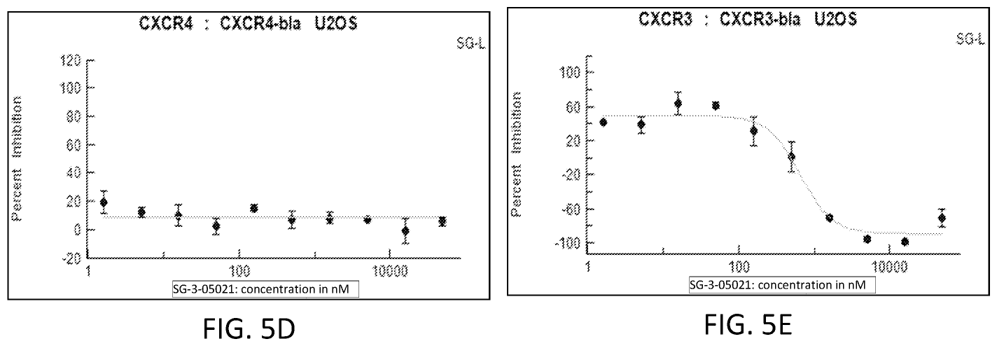

SG-3-05021 showed no agonistic activity for CCR7 (FIG. 5A), CXCR3 (FIG. 5B), or CXCR4 (FIG. 5C). SG-3-05021 showed no antagonistic activity for CXCR4 (FIG. 5D), and reverse antagonistic activity (sensitization) for CXCR3 (FIG. 5E).

To assay positive allosteric modulator activity of SG-3-00802 (SEQ ID NO: 1) and SG-3-006683 in CXCR3 cell lines, the cell line Tango™ CXCR3-bla U2OS was used in a positive allosteric modulator assay with two different methods, as described below.

Tango™ CXCR3-bla U2OS cells contain the human chemokine (C-X-C motif) receptor 3 linked to a TEV protease site and a Ga14-VP16 transcription factor stably integrated into the Tango™ GPCR-bla U2OS cell line. This parental cell line stably expresses a beta-arrestin/TEV protease fusion protein and the beta-lactamase reporter gene under the control of a UAS response element. The Tango™ CXCR3-bla U2OS cells have been functionally validated for a response to I-TAC (CXCL11).

In positive allosteric modulator assay method 1 (PAM1), cells were diluted in Assay Media to an appropriate cell density and were added to the assay plate. Peptides were added at increasing concentrations (1 nM-20 mcM) to the cells in the assay plate and incubated for 30 minutes. Ligand (CXCL9, CXCL10, or CXCL11) was added at an EC20 to all wells containing test compound. The assay plate was incubated for 16 hours at 37° C. Substrate Loading Solution was added to the assay plate and incubated for 2 hours at room temperature, in the dark. The assay plate was read on a fluorescence plate reader and the data was analyzed. PAM activity was calculated as follows:

% of PAM=((Test compound−Unstimulated)/(Stimulated−Unstimulated))×100.

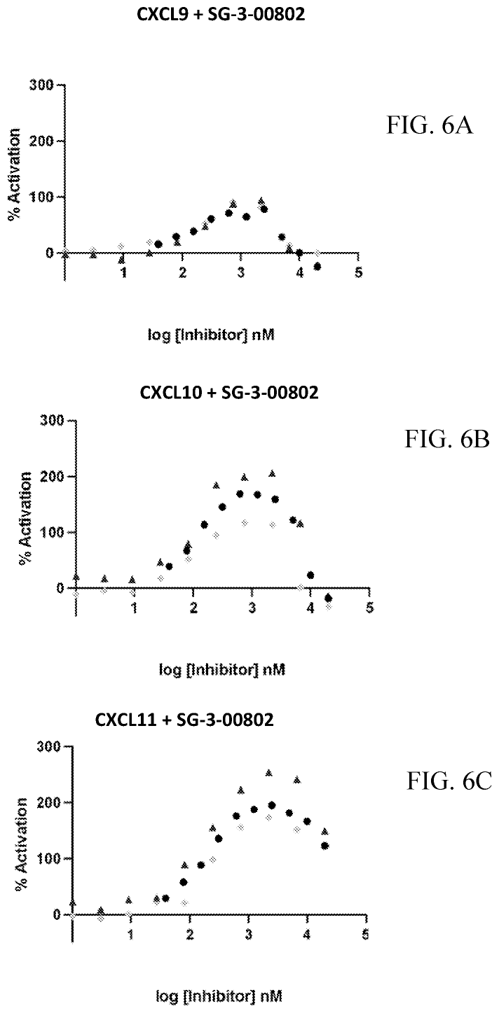

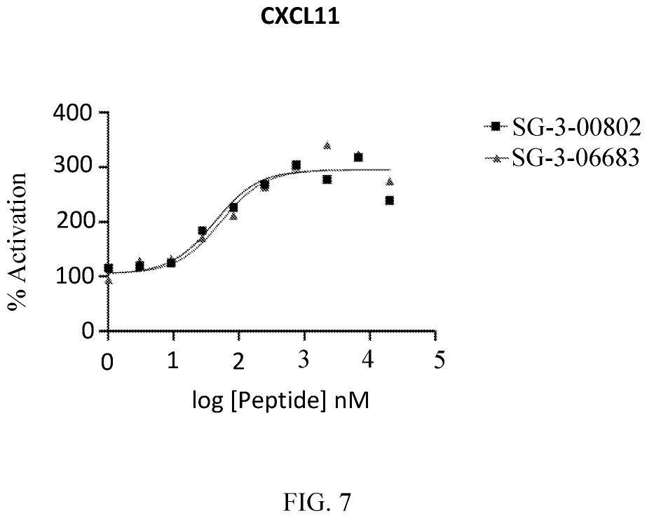

The addition of peptide SG-3-00802 (SEQ ID NO: 1) enhanced activity of CXCR3 in presence of its innate ligands CXCL9 (FIG. 6A), CXCL10 (FIG. 6B), and CXCL11 (FIG. 6C), indicating positive allosteric modulation. FIGS. 6A-6C show the percent activation of CXCR3 over stimulation with CXCL9, CXCL10, or CXCL11 ligand alone. Dose response curves are shown for 3 independent experiments. Additionally, a comparison of the activation of SG-3-00802 and SG-3-06683 showed similar positive allosteric modulation (FIG. 7).

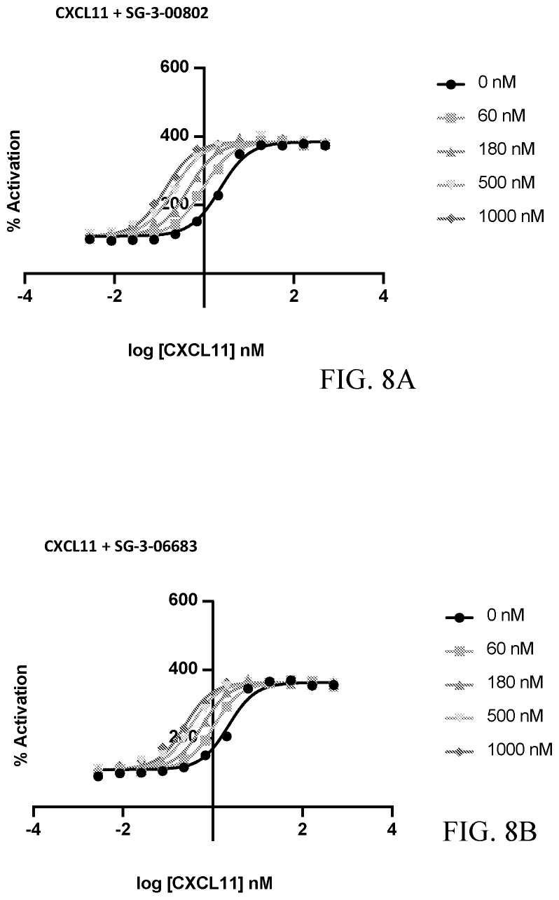

Positive allosteric modulation was confirmed with an additional test method using a dose response setting for CXCL11. In positive allosteric modulator assay method 2 (PAM2), cells were diluted in Assay Media to an appropriate cell density, and were added to the assay plate. CXCR3-bla U2OS were stimulated with varying concentrations of CXCL11 ligand and a pre-determined fixed concentration of either SG-3-00802 or SG-3-06683 and incubated for 30 minutes. Ligand (CXCL9, CXCL10, or CXCL11) was added at increasing concentrations to wells containing the test compound. SG-3-00802 and SG-3-06683 enhanced the activity of the endogenous ligand CXCL11 on the CXCR3 receptor when assayed in the PAM2 method (FIG. 8A-8B).

### Example 4. Prediction of SG-3-00802 Binding to Dendritic Cells

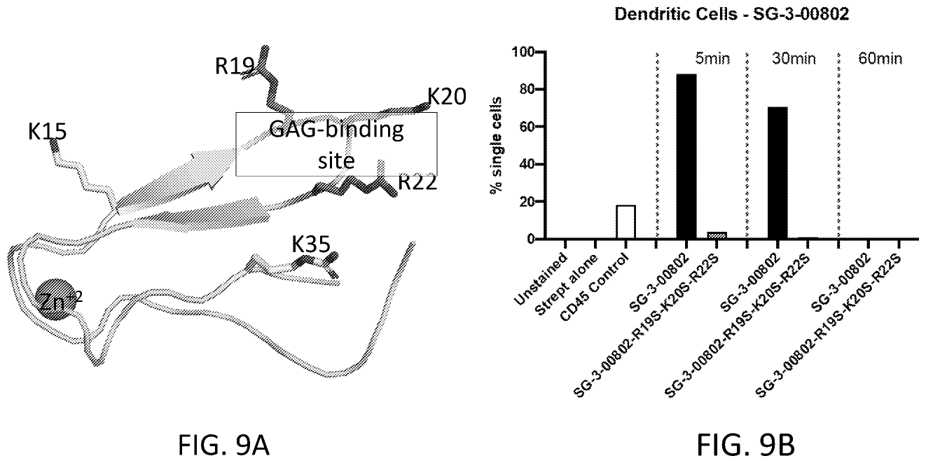

A predicted structure of SG-3-00802 was created by homology modeling using SWISS-MODEL (template PDB ID: 1DFE) and was superimposed with a homologous structure (PDB ID: 6FGP). The BBXB motif in glycosaminoglycans was present (FIG. 9A) (GAG-binding site, R19, K20, R22, are labeled). Positively charged residues (K15 and R35) could bind to the BBXB motif to form a cluster that could bind negatively charged GAG efficiently. The gray sphere indicates a zinc 2⁺ ion.

The binding of parental SG-3-00802 and SG-3-00802 where the GAG-binding site was mutated (SG-3-00802-R19S-K20S-R22S) to human dendritic cells was tested by flow cytometry. Mutation of the R19, K20S, R22S amino-acids that comprise the GAG-binding motif abolished binding of mutant SG-3-00802 to dendritic cells (FIG. 9B).

### Example 5. Assaying Cytokine Release In Vitro by SG-3-00802 and SG-3-05021

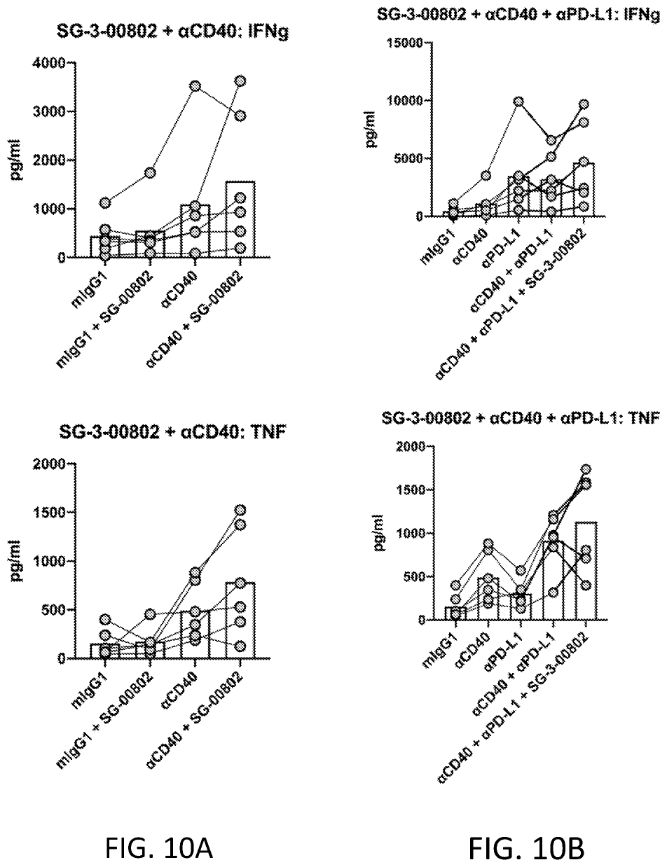

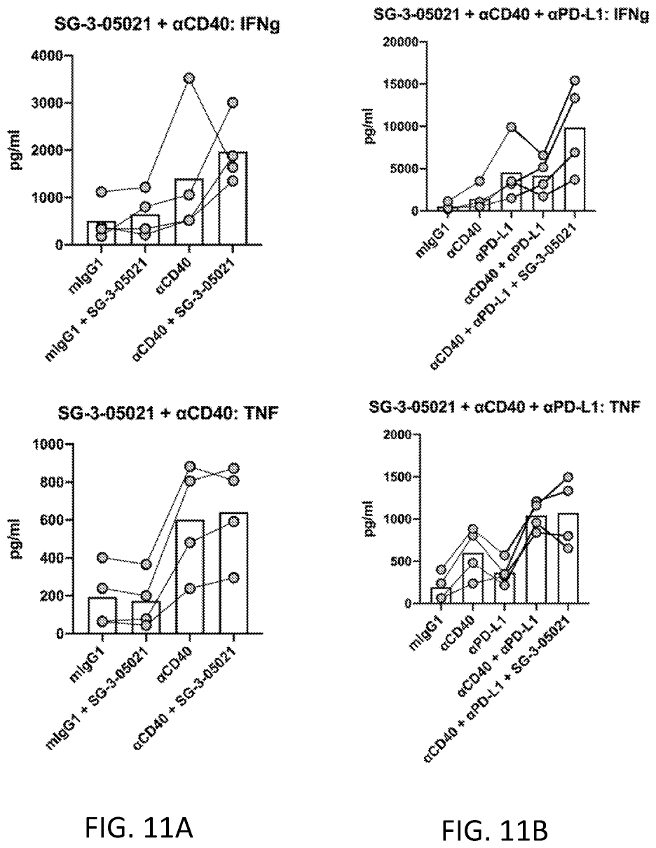

To demonstrate increase in activity in a mixed lymphocyte reaction (MLR) assay, immature monocyte-derived dendritic cells (moDCs) were pre-stimulated with anti-CD40 agonist antibody or the mouse IgGI isotype control antibody. Twenty-four hours later, T cells isolated from allogeneic donors were added to the moDCs with the peptide of interest, SG-3-00802 or SG-3-05021, alone or in combination with anti-PD-L1 monoclonal antibody. The co-culture of cells was incubated for 72 hours, after which cell supernatants were harvested and analyzed for secretion of cytokines, IFN-g and TNF-α. SG-3-05021 induced an increase in IFN-γ after stimulation with anti-CD40 either alone or in combination with anti-PD-L1 (FIGS. 10A-10B, 11A-11B). Data shown for experiment conducted in >4 Donor PBMCs.

### Example 6. Assaying Anti-Tumor Activity of SG-3-00802 in Combination with Anti-PD-1 Antibody In Vivo

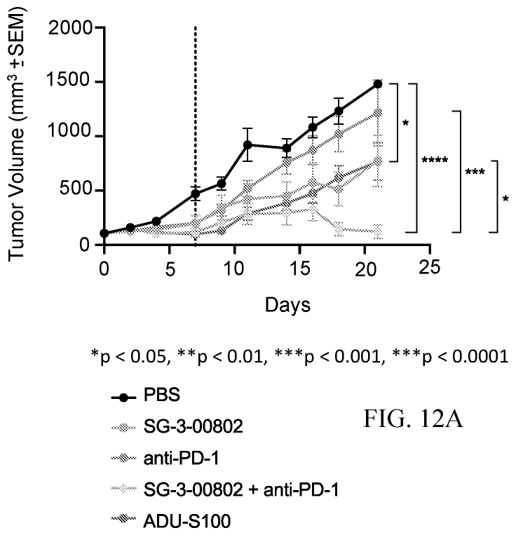

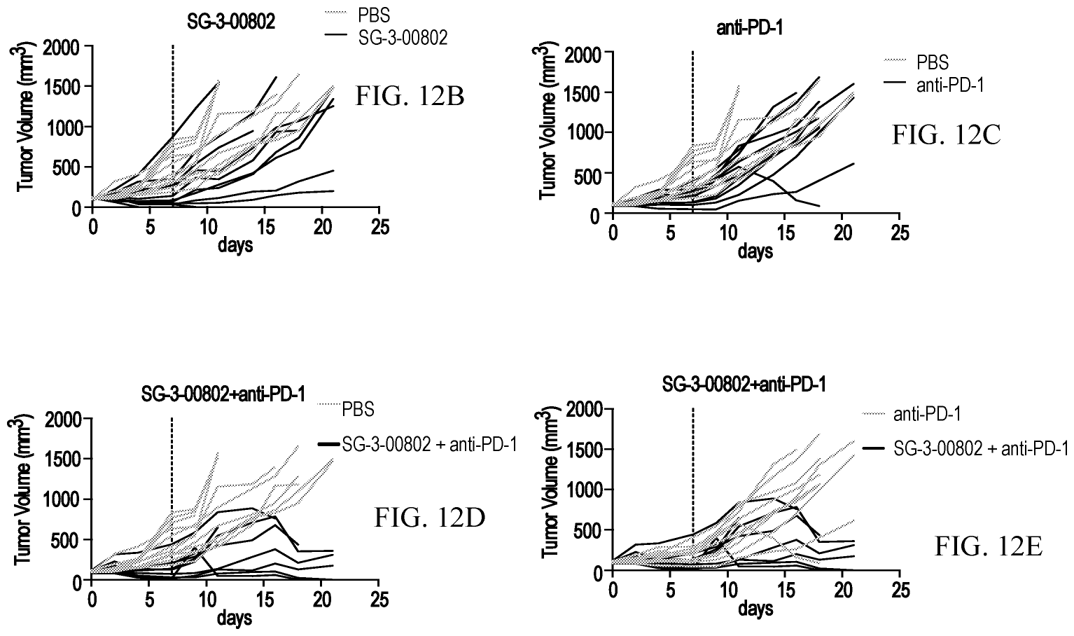

To determine anti-tumor activity of SG-3-00802 in vivo, the RENCA murine adenocarcinoma model was used. It is a syngeneic, standardized experimental model of metastatic RCC. Briefly, BALB/c female mice were inoculated subcutaneously with RENCA cells. Established tumors were treated daily with 2.5 mg/kg SG-3-00802 peritumorally, alone or in combination with intraperitoneal administration of 10 mg/kg anti-PD-1 antibody. A robust decrease in tumor volume either alone or in combination with anti-PD-1 compared to the phosphate buffered saline (PBS) treated control mice was observed over time, where time is in days after dosing initiation (FIGS. 12A-12E).

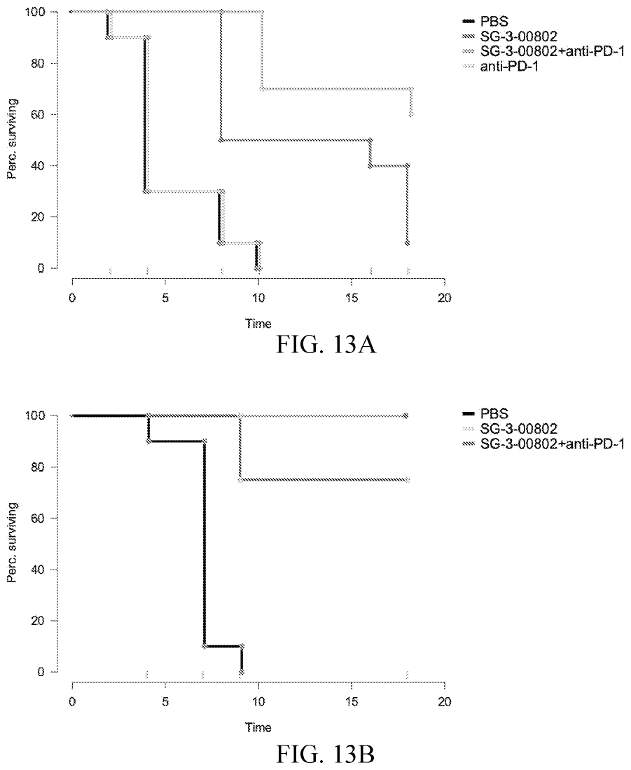

Additional studies were performed to test survival and tumor volume responses to a re-challenge event. In one study, mice were allocated into dosing groups when tumors reached ˜100 mm³. During the initial dosing study, time 0 was the first day of seven days of dosing. Initial dosing showed increased survival with either SG-3-00802 peptide or SG-3-00802 peptide in combination with anti-PD-1 antibodies (FIG. 13A). Re-implantation was completed on day 39 after dosing completion. Treated mice were tumor free for >20 days before being re-challenge. RENCA cells were implanted on the opposite flank (right flank) of the previously tumor bearing Balb/c mice and mice that did not receive any previous treatment were used as control. Tumor volume and survival was monitored as previously described. In the re-challenge experiment, time 0 is day 10 after re-challenge dosing. Re-challenge showed an increased percent of mice surviving after dosing with SG-3-00802 alone or in combination with anti-PD-1 antibody treatment (FIG. 13B).

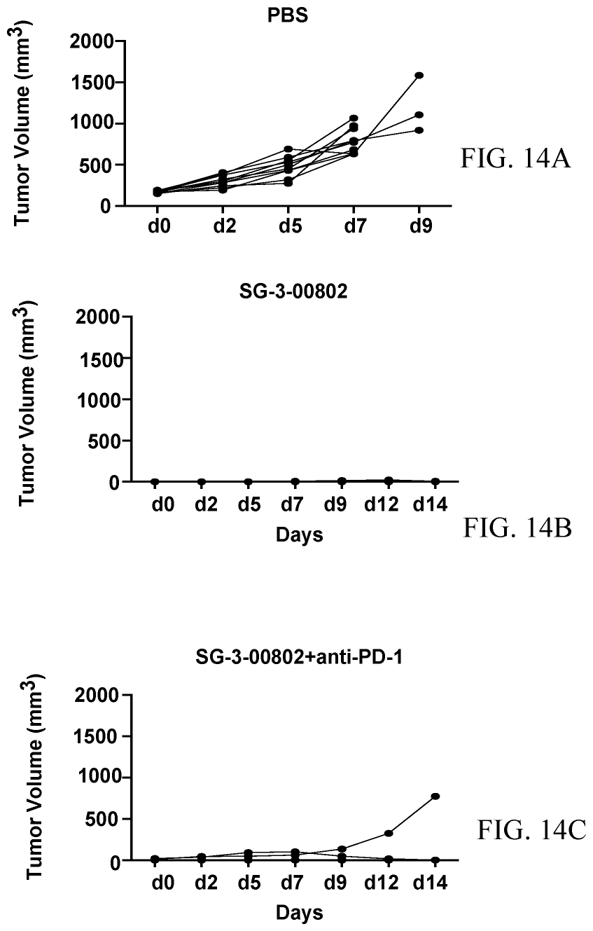

After a re-challenge, most animals of SG-3-00802 alone or SG-3-00802 with anti-PD-1 antibody combinatorial treatment rejected newly implanted tumors throughout the study (FIGS. 14A-14C). Results are summarized in Table 8.

TABLE 8
Summary of tumor-free animals in re-challenge study.

| | | # of animals |
|---|---|---|
| | # of animals with | without tumor |
| | complete tumor | progression out of |
| | regression out of | re-challenged |
| Treatment | total animals per | animals per group |
| Group | group (primary study) | (re-challenge study) |
| PBS | 0/12 | 0/10* |
| SG-3-00802 | 2/12 | 2/2 |
| (SEQ ID NO: 1) | | |
| Anti-PD-1 | 0/10 | n/a |
| SG-3-00802 | 4/12 | 3/4 |
| (SEQ ID NO: 1) + | | |
| Anti-PD-1 | | |

*newly implanted tumors in tumor naïve animals

### Example 8. Assaying Anti-Tumor Activity of SG-3-05021 in Combination with Anti-PD-1 Antibody In Vivo

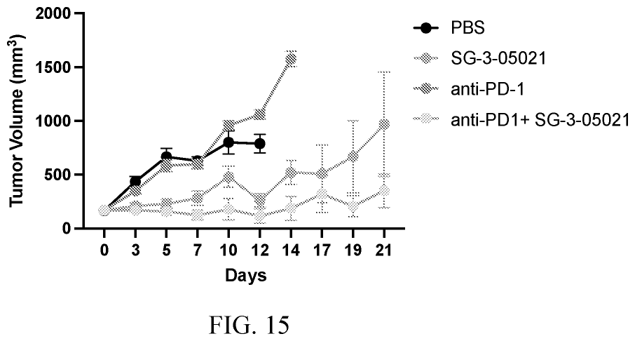

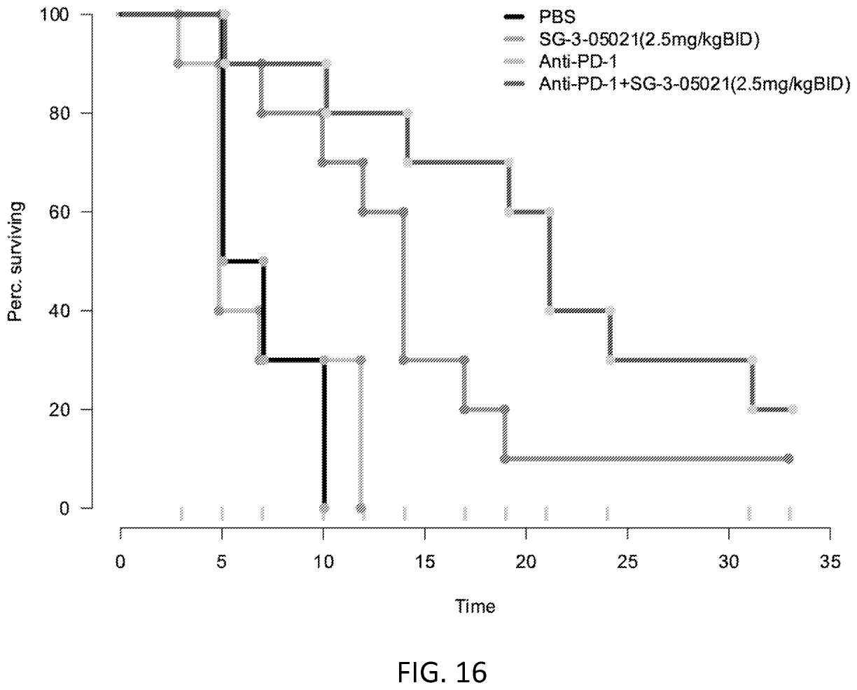

To determine anti-tumor activity of SG-3-05021 in vivo, the RENCA murine adenocarcinoma model was used. Briefly, BALB/c female mice were inoculated subcutaneously with RENCA cells. Established tumors were treated daily with 2.5 mg/kg SG-3-05021 peritumorally, alone or in combination with intraperitoneal administration of 10 mg/kg anti-PD-1 antibody. There was a decrease in tumor volume either alone with SG-3-05021 or in combination with anti-PD-1 (FIG. 15). Mice survived a longer time (days) following treatment with either the SG-3-05021 either alone or with anti-PD-1 antibody therapy compared to mice treated with phosphate buffered saline (PBS) or anti-PD-1 antibody therapy alone (FIG. 16).

### Example 9. Identifying Additional Human Targets of SG-3-00802 and SG-3-05021

The PathHunter® β-Arrestin Assay monitored activation of a panel of 168 G-protein coupled receptors (GPCRs) with fluorescent activation of the GPCR in agonist and antagonist mode.

$$\% {Activity}_{( Agonist mode )} = 100 * ( \frac{Mean R L {U}_{( test sample )} - Mean R L {U}_{( vehicle control )}}{Mean {MAX}_{( control ligand )} - Mean R L {U}_{( vehicle control )}} )$$

$$\% {Inhibition}_{( Antagonist mode )} = 100 * ( 1 - \frac{Mean R L {U}_{( test sample )} - Mean R L {U}_{( vehicle control )}}{Mean R L {U}_{( EC 80 control ligand )} - Mean R L {U}_{( vehicle control )}} )$$

where the test sample is the peptide, the vehicle control is DMSO (0% activity), and the control ligand is control compound (100% activity).

Peptides SG-3-00802 and SG-3-05021 were identified as an agonist, an antagonist, a PAM (Positive Allosteric Modulator), or an inverse-agonist. Agonist, antagonist, PAM, and inverse-agonist were identified according to the guidelines in Table 8. The basal activity/noise is a % activity of −20% to 20% for both agonist and antagonist modes. A PAM (Positive Allosteric Modulator) binds to a receptor, at a different site than the agonist, to change the receptor's response to the agonist. An inverse-Agonist is a binding partner with agonistic shutting down of basal activity in the cell. An antagonist antagonizes the activity of a particular binding partner.

CXCR3 and CXCR4 (DualSystems and Thermo SelectScreen assays) confirmed as hits for SG-3-00802. Six additional putative targets for SG-3-00802 were identified as were eleven putative targets for SG-3-05021 (see Tables 9-12).

TABLE 9
GPCR assay interpretation guideline

| % Agonist Activity | % Antagonist Activity | Interpretation |
|---|---|---|
| >25-30% | basal activity/noise | Agonist |
| basal activity/noise | >50% | Antagonist |
| >25-30% | —% (see below for exceptions) | Confirmation of positive agonist |
| >25-30% | —% (larger negative number = | PAM |
| | more likely) | |
| basal activity/noise | ~−30% | PAM |
| basal activity/noise | −70.8 | strong PAM |
| −25 to −30%-−80% | >50% | Inverse-Agonist |

TABLE 10
SG-3-00802 target assay results.

| SG-3-00802 | |
|---|---|
| gpcrMAX | SelectScreen |

| | Agonist | Antagonist | Agonist | Antagonist |
|---|---|---|---|---|
| Gene | Mode | Mode | Mode | Mode |
| CCR7 | 0% | 0% | No | NA |
| CXCR3 | −4% | −32% | No | PAM |
| CXCR4 | −14% | 99% | No | Yes |

TABLE 11
SG-3-00802 assay results.

| | % Agonist | % Antagonist | |
|---|---|---|---|
| Gene | Activity | Activity | Interpretation |
| CCR7 | 0% | 0% | No |
| CCR9 | 2% | −48% | PAM |
| CXCR3 | −4% | −32% | PAM |
| CXCR4 | −14% | 99% | Antagonist |

TABLE 12
SG-3-05021 target assay results

| | SG-3-05021 | | | |
|---|---|---|---|---|
| | gpcrMAX | | SelectScreen | |
| | Agonist | Antagonist | Agonist | Antagonist |
| Gene | Mode | Mode | Mode | Mode |
| CCR7 | 0% | −18% | No | NA |
| CXCR3 | −4% | −20% | No | PAM |
| | | | | (<potent than 802) |
| CXCR4 | 1% | 10% | No | NO |

### Example 11. Using Informatics to Identify Sequences Related to Peptide SG-3-00802

Additional sequences identified with the informatics pipeline include: SEQ ID NOs: 47-50 (see Table 13). The informatics pipeline included analyzing a large and curated microbiome database of subjects with cancer treated with anti-PD-1. Microbiome derived peptides associated with subject response to anti-PD-1 treatment were identified and tested for binding and activity with immune cell phage display and cell-based assays.

Meta-analysis of multiple microbiome studies in the context of anti-PD-1 response was conducted to identify significantly enriched bacterial strains in anti-PD-1 responders. Cohorts included patients with melanoma or pan-cancer (melanoma, renal cell carcinoma, and non-small cell lung cancer) undergoing ICI therapy. Patients preferably had response data available at 6 months after start of therapy; however, response data at 3 months were used if 6-month response data were not available. Stool samples in these studies were collected from patients prior to the start of ICI therapy. Exclusion criteria included patients whose tumors were surgically resected prior to the start of ICI therapy and patients in whom the ICI drug was switched during the course of treatment.

Based on meta-analysis of published datasets and characterization of baseline microbiome of anti-PD-1 antibody-treated melanoma patients in previous studies, we identified 37 unique bacterial strains enriched in anti-PD-1 responders vs non-responders concordant across multiple cohorts. The meta-analysis also identified novel strains that were not reported in original studies.

Next, peptides from strains associated with responder signatures were predicted from their genome sequences. In addition, we predicted peptides from assembled metagenomes that were associated with responders. The predicted 90,000+ peptides were screened using phage display technology to identify binders to immune cells (T cells, peripheral blood mononuclear cells, monocytes and DCs) known to play a role in the tumor microenvironment.

Peptides that bound to specific immune cells (1% of the screened peptides) were then evaluated for activity in cell-based assays using isolated primary human T cells, DCs, and macrophages. Immune-driven inhibition of tumor growth relies on DC-mediated recruitment of effector T cells, mainly via CXCL9 and CXCL10. Hence, induction of CXCL10 was selected as a screening readout to identify DC-targeted peptides that can modulate effector cell recruitment. Peptides inducing robust CXCL10 secretion (LPS)-stimulated human monocyte-derived DCs from multiple donors, with a mean induction of >2-fold of CXCL10 over OVA323 339 control peptide in DCs were selected for dose response analysis (n=36). Of these selected DC-targeting peptides, 15 demonstrated robust dose-dependent response in vitro in the same assay setup (including SG-3-00802 and SG-3-005021). To confirm cross species activity, 10 of the 15 peptides with EC50<10 μM were screened in vitro for CXCL10 induction in mouse bone marrow DCs.

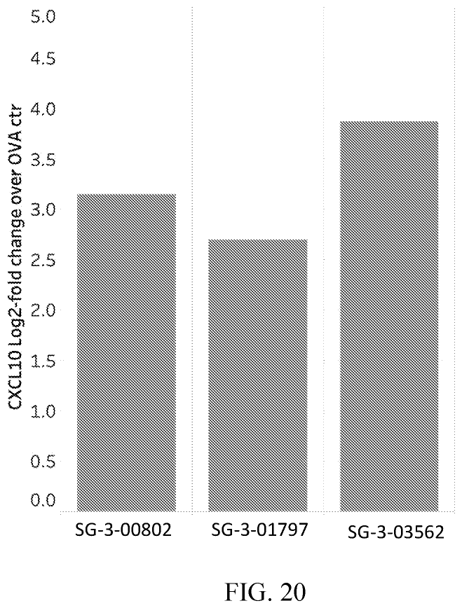

A sequence alignment of SEQ ID Nos 1, and 47-50 is shown in Table 13. The homologous peptides have been shown to bind to primary immune cells via phage display. SG-3-00802 homologous peptides that were tested in the dendritic cell CXCL10 release assay described in Example 1 show similar activity to SG-3-00802 (FIG. 20).

TABLE 13
Alignment of sequences.

| | | | Sequence |
|---|---|---|---|
| | | | Similarity** |
| Sequence | Peptide | | to SEQ |
| Identifier | Description | Sequence* | ID NO: 1 |
| SEQ ID | SG-3-00802 | MKVRPSVKPMCEKCKIIRRKGRVMVICENPKHKQRQ | 100% |
| NO: 1 | | G | |
| SEQ ID | SG-3-01677 | MKVRsSVKPICEKCKVIKRKGaIrVICENPKHKQRQG | 86.7% |
| NO: 47 | | | |
| SEQ ID | SG-3-01797 | MKVRPSVKkMCDKCKIIRRHGKVLVICENPRHKQRQ | 86.8% |
| NO: 48 | | G | |
| SEQ ID | SG-3-03562 | MKVRPSVKPMCDKCRVIKRKGRVMIICENPKHKQR | 94.3% |
| NO: 49 | | QG | |
| SEQ ID | SG-3-03785 | MKVRPSVKPMCEKCKIIKRKGKVMVICENPKHKQR | 97.0% |
| NO: 50 | | QG | |

*Lower-case letters represent dissimilar residues compared to SG-3-00802, Bold represents similarresidues compared to SG-3-00802 substitution for amino acids.
**Sequence similarity calculation below is based on the BLOSUM62 substitution for amino acids.

### Example 10. In Vitro Migration of Peptide SG-3-00802 and Variants Thereof

T cell migration towards CXCR3 ligands in the presence of SG-3-00802 and SG-3-06683 was tested using a Transwell migration assay. Suboptimal concentrations of each ligand (CXCR3 ligands CXCL9, CXCL10, and CXCL11) were added to the bottom of the transwell with increasing doses of SG-3-00802 or SG-3-006683. An optimal concentration of ligand was used as positive control, as indicated in the graphs.

Unstimulated human T cells were resuspended in AIM V serum-free media, then were added to the top chamber of a 5 μM pore polycarbonate Transwell insert and incubated with the indicated concentrations of the ligands and peptides in the bottom chamber for 2.5 hours at 37° C. T cells that migrated to the bottom chamber were collected, stained with anti-CD3 antibody, and counted using Beckman Coulter Cytoflex flow cytometer. 123count eBeads counting beads were added to each of the wells for obtaining the absolute cell counts.

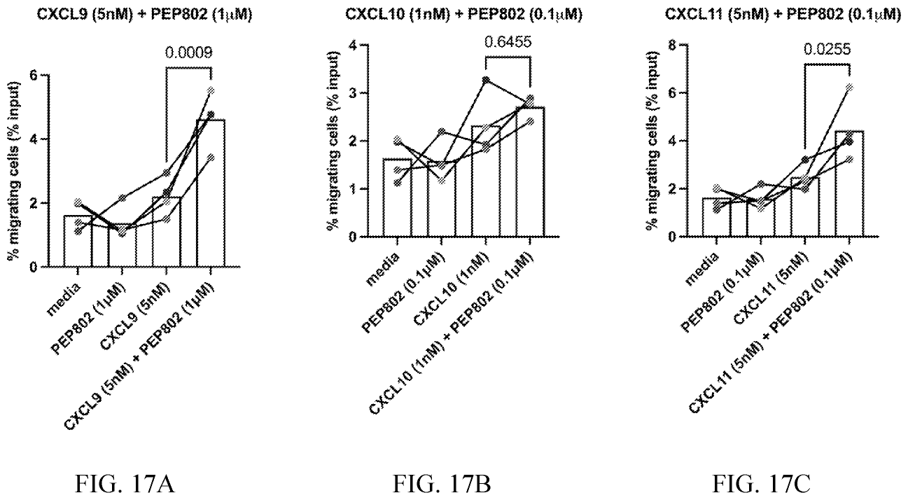

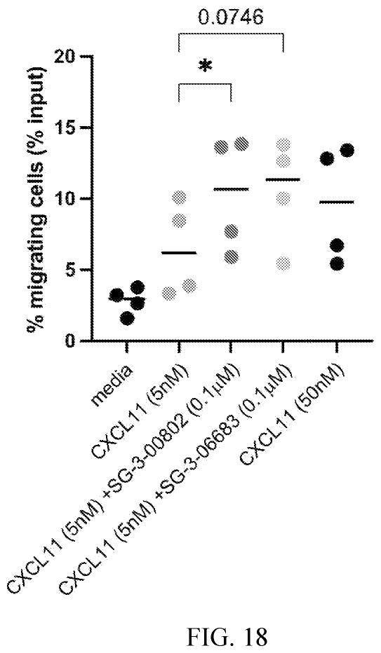

SG-3-00802 increased migration to suboptimal levels of all 3 CXCR3 ligands, CXCL9 (FIG. 17A), CXCL10 (FIG. 17B), and CXCL11 (FIG. 17C), supporting positive allosteric modulation (PAM) of CXCR3 in a functional assay. Additionally, variants of SG-3-00802, specifically SG-3-006683 (SEQ ID NO: 46), increased migration to suboptimal levels of CXCL11 (FIG. 18), supporting positive allosteric modulation (PAM) of CXCR3 in a functional assay.

### Example 11. SG-3-00802 Increases CXCL10 Levels in the Serum and Tumor of Tumor Bearing Mice

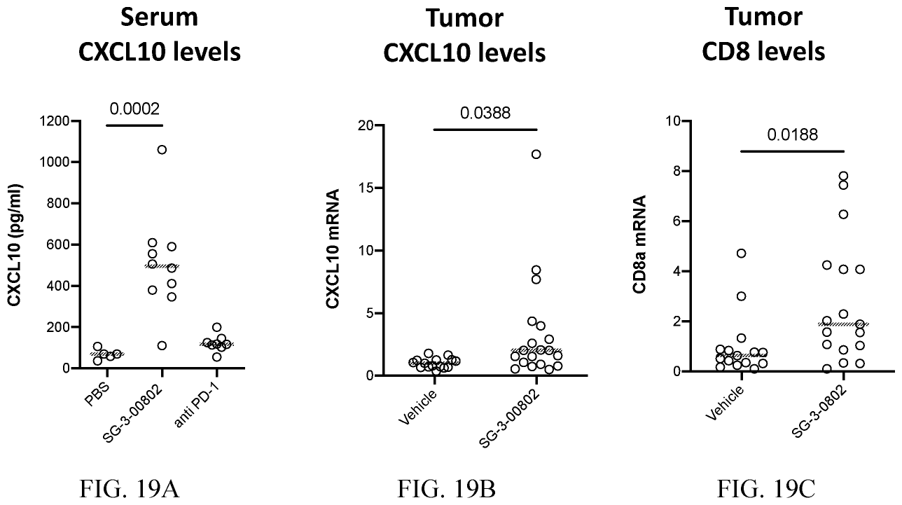

Several mechanism of action related endpoints were also measured in vivo. Balb/c mice were implanted with RENCA cells and treated with candidate lead peptides and control compounds. Dosing occurred during the 1st week only: SG-3-00802 was dosed peritumorally at 2.5 mg/kg BID for 7d, anti-PD-1 was dosed i.p. at 10 mg/kg TIW for a total of 3 doses. Treatment with SG-3-00802 in the RENCA tumor model increased serum levels of CXCL10 (FIG. 19A), which makes CXCL10 a promising PD biomarker for the clinical setting. Treatment with SG-3-00802 also increased expression of CXCL10 in the tumor (FIG. 19B), which correlated with an increase in CD8a expression (FIG. 19C), indicating that SG-3-00802-mediated CXCL10 induction enhances recruitment of effector cells into the tumor.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A peptide comprising an amino acid sequence having at least 85% sequence identity to any one of SEQ ID NOs: 1-65, preferably SEQ ID NO:46 (GQRQKHKPNECIVMVRGKRRIIKCKECMPKVSPRVKM) or SEQ ID NO:51 (LGKTDWIEKYFKVKKEKIDKMQRFLQG).

2. The peptide of claim 1, wherein the peptide comprises an amino acid sequence having at least 90%, at least 95%, or at least 99% sequence identity to any one of SEQ ID NOs: 1-65, preferably SEQ ID NO:46 (GQRQKHKPNECIVMVRGKRRIIKCKECMPKVSPRVKM) or SEQ ID NO:51 (LGKTDWIEKYFKVKKEKIDKMQRFLQG).

3. The peptide of claim 1 or claim 2, wherein the peptide comprises the amino acid sequence any one of SEQ ID NOs: 1-65, preferably of SEQ ID NO: 46 (GQRQKHKPNECIVMVRGKRRIIKCKECMPKVSPRVKM) or SEQ IDNO: 51 (LGKTDWIEKYFKVKKEKIDKMQRFLQG).

4. The peptide of any one of claims 1-3, wherein the peptide comprises at least one D-amino acid.

5. The peptide of any one of claims 1-4, wherein the peptide consists of D-amino acids.

6. A nucleic acid construct comprising a polynucleotide, wherein the polynucleotide encodes a peptide of any one of claims 1-5.

7. A recombinant host cell comprising an exogenous polynucleotide, the polynucleotide encoding a peptide having at least 85%, at least 90%, at least 95%, or at least 99%, or 100% sequence identity to any one of SEQ ID NOs: 1-65.

8. The recombinant host cell of claim 7, wherein the exogenous polynucleotide further encodes a host cell specific signal sequence.

9. The recombinant host cell of claim 7 or claim 8, wherein the exogenous polynucleotide further encodes a heterologous promoter.

10. The recombinant host cell of claim 9, wherein the heterologous promoter is a constitutive promoter.

11. The recombinant host cell of claim 9, wherein the heterologous promoter is an inducible promoter.

12. The recombinant host cell of any one of claims 7-11, wherein the recombinant host cell is a prokaryotic cell, an eukaryotic cell, or a fungal cell.

13. The recombinant host cell of claim 12, wherein the recombinant host cell is selected from the group consisting of: an Escherichia coli cell, a Lactococcus lactis cell, a Streptomyces coelicolor cell, a Streptomyces lividans cell, a Streptomyces albus cell, a Streptomyces venezuelae cell, or a Bacillus subtilis cell.

14. The recombinant host cell of claim 12, wherein the recombinant host cell is a Saccharomyces cerevisiae cell, a Pichia pastoris cell, a Yarrowia lipolytica cell, an Aspergillus niger cell, or a Hansenula polymorpha cell.

15. The recombinant host of claim 12, wherein the eukaryotic cell is a Chinese Hamster Ovary cell.

16. A pharmaceutical composition comprising:
a) a peptide having at least 85%, at least 90%, at least 95%, or at least 99%, or 100% sequence identity to any one of SEQ ID NOs: 1-65, the nucleic acid construct of claim 6, or a plurality of recombinant host cells of any one of claims 7-15; and
b) a pharmaceutically acceptable carrier.

17. The pharmaceutical composition of claim 16, wherein the pharmaceutical composition is formulated for oral administration, rectal administration, intravenous administration, or intratumoral administration.

18. The pharmaceutical composition of any one of claims 16-17, wherein the pharmaceutical composition is formulated as a tablet, a capsule, a powder, or a liquid.

19. The pharmaceutical composition of claim 18, wherein the composition is formulated as a tablet.

20. The pharmaceutical composition of claim 19, wherein the tablet is coated.

21. The pharmaceutical composition of claim 20, wherein the coating comprises an enteric coating.

22. A method of modulating the activity of one or more target proteins in a subject, the method comprising administering to the subject:
a) a peptide having at least 85% sequence identity to any one of SEQ ID NOs: 1-65, and/or
b) the peptide of any one of claims 1-5, and/or
c) the nucleic acid construct of claim 6, and/or
d) a plurality of recombinant host cells of any one of claims 7-15, and/or e) the pharmaceutical composition of any one of claims 16-21;
wherein the one or more target proteins are selected from the group consisting of a CCR9 protein, a CXCR3 protein, and a CXCR4 protein.

23. The method of claim 22, wherein the CCR9 protein and/or the CXCR3 protein has an activity that is increased relative to a subject that did not receive treatment or relative to a reference value.

24. The method of claim 22, wherein the CXCR4 protein has an activity that is decreased relative to a subject that did not receive treatment or relative to a reference value.

25. The method of claim 23 or 24, wherein the activity is measured with a beta-arrestin reporter cell assay.

26. A method for treating a disease in a subject in need thereof, the method comprising administering to the subject:
a) a peptide having at least 85% sequence identity to any one of SEQ ID NOs: 1-65, and/or
b) the peptide of any one of claims 1-5, and/or
c) the nucleic acid construct of claim 6, and/or
d) a plurality of recombinant host cells of any one of claims 7-15, and/or
e) the pharmaceutical composition of any one of claims 16-21.

27. The method of claim 26, wherein the disease is a neoplasm.

28. The method of claim 26 or claim 27, wherein the disease is cancer.

29. The method of claim 28, wherein the cancer is at least one selected from the group consisting of: melanoma, renal cell carcinoma, non-small cell lung carcinoma, basal cell carcinoma, biliary tract cancer, bladder cancer, bone cancer, brain and central nervous system cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, small-cell lung cancer, Hodgkin's lymphoma, non-Hodgkins lymphoma, myeloma, neuroblastoma, oral cavity cancer, ovarian cancer, pancreatic cancer, prostate cancer, retinoblastoma, rhabdomyosarcoma, rectal cancer, cancer of the respiratory system, sarcoma, skin cancer, stomach cancer, testicular cancer, thyroid cancer, uterine cancer, and cancer of the urinary system.

30. A method for increasing the response to an immunotherapy in a subject in need thereof comprising administering to the subject a composition, wherein the composition comprises:
i) a peptide having at least 85% sequence identity to any one of SEQ ID NOs: 1-65, and/or
ii) the peptide of any one of claims 1-5, and/or
iii) the nucleic acid construct of claim 6, and/or
iv) a plurality of recombinant host cells of any one of claims 7-15, and/or
v) the pharmaceutical composition of any one of claims 16-21.

31. The method of any one of claims 26-30, wherein the peptide modulates the production of at least one cytokine in the subject relative to a reference value.

32. The method of claim 31, wherein the cytokine is selected from the group consisting of TNF-α, IL-17, IL-1β, IL-2, IFN-γ, IL-6, IL-12, IL-25, IL-33, IL-8, MCP-1, MIP-3α, CXCL1, IL-23, IL-4, IL-10, IL-13, IFN-α, and TGF-β.

33. The method of any one of claims 26-32, wherein the peptide induces the production of at least one pro-inflammatory cytokine in the subject relative to a reference value.

34. The method of claim 33, wherein the at least one pro-inflammatory cytokine is selected from the group consisting of TNF-α, IL-17, IL-1β, IL-2, IFN-γ, IL-6, IL-12, IL-25, IL-33, IL-8, MCP-1, MIP-3α, CXCL1, and IL-23.

35. The method of any one of claims 26-34, wherein the peptide suppresses the production of at least one anti-inflammatory cytokine in the subject relative to a reference value.

36. The method of claim 35, wherein the at least one anti-inflammatory cytokine is selected from the group consisting of IL-4, IL-10, IL-13, IFN-α, and TGF-β.

37. The method of any one of claims 26-36, wherein the peptide increases Th1 activation in the subject relative to a reference value.

38. The method of any one of claims 26-37, wherein the peptide increases dendritic cell maturation in the subject relative to a reference value.

39. The method of any one of claims 26-38, wherein the peptide increases CD70 expression in the subject relative to a reference value.

40. The method of any one of claims 26-39, wherein the peptide increases the clonal expansion of T_{eff} in the subject relative to a reference value.

41. The method of any one of claims 26-40, wherein the peptide increases activity of a CCR9 protein or a CXCR3 protein relative to a reference value.

42. The method of any one of claims 26-41, wherein the peptide decreases activity of a CXCR4 protein relative to a reference value.

43. The method of any one of claims 26-42, wherein the peptide binds to a CCR9 protein, a CXCR3 protein, or a CXCR4 protein.

44. The method of any one of claims 26-43 further comprising administering an additional treatment for cancer and/or other adjunct therapy to the subject.

45. A method of treating cancer in a subject in need thereof, the method comprising:
a) administering to the subject:
i) a peptide having at least 85% sequence identity to any one of SEQ ID NOs: 1-65, and/or
ii) the peptide of any one of claims 1-5, and/or
iii) the nucleic acid construct of claim 6, and/or
iv) a plurality of recombinant host cells of any one of claims 7-15, and/or
v) the pharmaceutical composition of any one of claims 16-21; and
b) administering to the subject an additional treatment for cancer and/or adjunct therapy to the subject.

46. The method of claim 44 or claim 45, wherein the additional treatment for cancer and/or adjunct therapy comprises a probiotic.

47. The method of claim 44 or claim 45, wherein the additional treatment for cancer and/or adjunct therapy comprises surgery, radiation therapy, or a combination thereof.

48. The method of claim 44 or claim 45, wherein the additional treatment for cancer and/or adjunct therapy comprises a therapeutic agent.

49. The method of claim 48, wherein the therapeutic agent comprises a chemotherapeutic agent, a targeted therapy, an additional immunotherapy, or a combination thereof.

50. The method of claim 49, wherein the chemotherapeutic agent comprises carboplatin, cisplatin, gemcitabine, methotrexate, paclitaxel, pemetrexed, lomustine, temozolomide, dacarbazine, or a combination thereof.

51. The method of claim 49, wherein the targeted therapy comprises afatinib dimaleate, bevacizumab, cetuximab, crizotinib, erlotinib, gefitinib, sorafenib, sunitinib, pazopanib, everolimus, dabrafenib, aldesleukin, interferon alfa-2b, ipilimumab, peginterferon alfa-2b, trametinib, vemurafenib, or a combination thereof.

52. The method of claim 49, wherein the additional immunotherapy comprises a cell therapy, a therapy with an immune checkpoint inhibitor, a therapy with a co-stimulatory immune checkpoint agent, or a combination thereof.

53. The method of claim 52, wherein the immune checkpoint inhibitor is selected from the group consisting of: ipilimumab, nivolumab, pembrolizumab, atezolizumab, avelumab, durvalumab, cemiplimab, and a combination thereof.

54. The method of claim 52, wherein the co-stimulatory immune checkpoint agent is selected from the group consisting of: IBI101, utomilumab, MEDI1873, and a combination thereof.

55. The method of claim 52, wherein the cell therapy is a CAR T cell therapy

56. The method of any one of claims 26-55, wherein the subject is a human.

57. A method of producing a peptide, the method comprising culturing the recombinant host cell of any one of claims 7-15, under conditions sufficient for expression of the encoded peptide.

58. A method of producing a peptide, the method comprising chemically synthesizing the peptide of any one of SEQ ID NOs: 1-65.


SEQUENCE LISTING (65 sequences)

SEQ ID NO: 1 - protein, 37 aa, Oscillibacter sp.
MKVRPSVKPMCEKCKIIRRKGRVMVICENPKHKQRQG

SEQ ID NO: 2 - protein, 37 aa, Unknown
  peptide for immunotherapy; SG-3-00802_variant 1 peptide
MKVRPSVKPICEKCKIIRRKGRVMVICENPKHKQKQG

SEQ ID NO: 3 - protein, 37 aa, Unknown
  peptide for immunotherapy; SG-3-00802_variant 2 peptide
MKVRPSVKPMCEKCKIIKRKGKVMVICENPKHKQRQG

SEQ ID NO: 4 - protein, 37 aa, Unknown
  peptide for immunotherapy; SG-3-00802_variant 3 peptide
MKVRPSVKPMCEKCKIIKRKGKVMVICENPKHKQKQG

SEQ ID NO: 5 - protein, 37 aa, Unknown
  peptide for immunotherapy; SG-3-00802_variant 4 peptide
MKVRPSVKPICEKCKIIRRKGRVMVICQNPKHKQKQG

SEQ ID NO: 6 - protein, 37 aa, Unknown
  peptide for immunotherapy; SG-3-00802_variant 5 peptide
MKVRPSVKPICEKCKIIKRKGRVMVICENPKHKQKQG

SEQ ID NO: 7 - protein, 37 aa, Unknown
  peptide for immunotherapy; SG-3-00802_variant 6 peptide
MKVRPSVKPMCEKCKVIKRKGKVMVICENPKHKQRQG

SEQ ID NO: 8 - protein, 37 aa, Unknown
  peptide for immunotherapy; SG-3-00802_variant 7 peptide
MKVRPSVKPMCEKCKVIKRKGRVMVICENPKHKQKQG

SEQ ID NO: 9 - protein, 37 aa, Unknown
  peptide for immunotherapy; SG-3-00802_variant 8 peptide
MKVRPSVKPICEKCKVIRRKGRVMVICENPKHKQKQG

SEQ ID NO: 10 - protein, 37 aa, Unknown
  peptide for immunotherapy; SG-3-00802_variant 9 peptide
MKVKPSVKPICEKCKIIKRKGRVMVICENPKHKQKQG

SEQ ID NO: 11 - protein, 37 aa, Unknown
  peptide for immunotherapy; SG-3-00802_variant 10 peptide
MKVRPSVKPMCEKCKVIKRKGKVMVICENPKHKQKQG

SEQ ID NO: 12 - protein, 37 aa, Unknown
  peptide for immunotherapy; SG-3-00802_variant 11 peptide
MKVRPSVKKICEKCKIIKRKGRVMVICENPKHKQKQG

SEQ ID NO: 13 - protein, 37 aa, Unknown
  peptide for immunotherapy; SG-3-00802_variant 12 peptide
MKIRPSVKPMCEKCRIIKRKGRVMVICENPKHKQKQG

SEQ ID NO: 14 - protein, 37 aa, Unknown
  peptide for immunotherapy; SG-3-00802_variant 13 peptide
MKVRPSVKPICEKCKVIRRKGKVMVICENPKHKQKQG

SEQ ID NO: 15 - protein, 37 aa, Unknown
  peptide for immunotherapy; SG-3-00802_variant 14 peptide
MKVRPSVKPICEKCKIIKRKGKVMVICENPKHKQKQG

SEQ ID NO: 16 - protein, 37 aa, Unknown
  peptide for immunotherapy; SG-3-00802_variant 15 peptide
MKVRPSVKPICEKCKIIRRKGKVMVICQNPKHKQKQG

SEQ ID NO: 17 - protein, 37 aa, Unknown
  peptide for immunotherapy; SG-3-00802_variant 16 peptide
MKVRPSVKPICEKCKVIKRKGRVMVICENPKHKQKQG

SEQ ID NO: 18 - protein, 37 aa, Unknown
  peptide for immunotherapy; SG-3-00802_variant 17 peptide
MKVKPSVKTICEKCKIIRRKGRVMVICENPKHKQKQG

SEQ ID NO: 19 - protein, 37 aa, Unknown
  peptide for immunotherapy; SG-3-00802_variant 18 peptide
MKVRPSVKPICEKCKVIKRKGKVMVICENPKHKQRQG

SEQ ID NO: 20 - protein, 38 aa, Unknown
  peptide for immunotherapy; SG-3-00802_variant 19 peptide
MKVRPSVKPICDKCRIIKRKGRVMVICENPKHKQRQGN

SEQ ID NO: 21 - protein, 37 aa, Unknown
  peptide for immunotherapy; SG-3-00802_variant 20 peptide
MKVKPSVKPICEKCKVIRRKGRVMVICQNPKHKQRQG

SEQ ID NO: 22 - protein, 37 aa, Unknown
  peptide for immunotherapy; SG-3-00802_variant 21 peptide
MKVKPSVKTICEKCKIIKRKGRVMVICENPKHKQKQG

SEQ ID NO: 23 - protein, 37 aa, Unknown
  peptide for immunotherapy; SG-3-00802_variant 22 peptide
MKVQPSVKKICEKCKIIKRKGRVMVICENPKHKQKQG

SEQ ID NO: 24 - protein, 37 aa, Unknown
  peptide for immunotherapy; SG-3-00802_variant 23 peptide
MKVRSSVKPICEKCKIIRRKGSIRVICENPKHKQRQG

SEQ ID NO: 25 - protein, 37 aa, Unknown
  peptide for immunotherapy; SG-3-00802_variant 24 peptide
MKVKPSVKPICEKCKVIKRKGRVMVICQNPKHKQRQG

SEQ ID NO: 26 - protein, 37 aa, Unknown
  peptide for immunotherapy; SG-3-00802_variant 25 peptide
MKVRSSVKPICEKCKIIKRKGRIRVICENPKHKQRQG

SEQ ID NO: 27 - protein, 37 aa, Unknown
  peptide for immunotherapy; SG-3-00802_variant 26 peptide
MKVRPSVKPICEKCKVIKRKGKVMVICENPKHKQKQG

SEQ ID NO: 28 - protein, 37 aa, Unknown
  peptide for immunotherapy; SG-3-00802_variant 27 peptide
MKVKPSVKKICEKCKIIKRKGRVMVICENPKHKQKQG

SEQ ID NO: 29 - protein, 37 aa, Unknown
  peptide for immunotherapy; SG-3-00802_variant 28 peptide
MKVRPSVKPICEKCKVIKRKGKVMVICQNPKHKQRQG

SEQ ID NO: 30 - protein, 37 aa, Unknown
  peptide for immunotherapy; SG-3-00802_variant 29 peptide
MKVKPSVKTICEKCKIIRRKGRVMIICENPKHKQKQG

SEQ ID NO: 31 - protein, 37 aa, Unknown
  peptide for immunotherapy; SG-3-00802_variant 30 peptide
MKVRPSVKPICEKCKVIKRKGHVMVICENPKHKQKQG

SEQ ID NO: 32 - protein, 37 aa, Unknown
  peptide for immunotherapy; SG-3-00802_variant 31 peptide
MKVRPSVKPICDKCRVIKRKGRVMVICENPKHKQRQG

SEQ ID NO: 33 - protein, 39 aa, Unknown
  peptide for immunotherapy; SG-3-00802_variant 32 peptide
MKVRSSVKPICEKCKIIRRKGSIRVICENPKHKQRQDRH

SEQ ID NO: 34 - protein, 38 aa, Unknown
  peptide for immunotherapy; SG-3-00802_variant 33 peptide
MKVRPSVKPICEYCKVIRRNGRVMVICPTNPKHKQRQG

SEQ ID NO: 35 - protein, 37 aa, Unknown
  peptide for immunotherapy; SG-3-00802_variant 34 peptide
MKVRSSVKPICEKCKIIKRKGSIRVICENPKHKQRQG

SEQ ID NO: 36 - protein, 37 aa, Unknown
  peptide for immunotherapy; SG-3-00802_variant 35 peptide
MKVKPSVKPICEKCKVIKRKGRVMIICANPKHKQRQG

SEQ ID NO: 37 - protein, 37 aa, Unknown
  peptide for immunotherapy; SG-3-00802_variant 36 peptide
MKVKPSVKTICEKCKIIKRKGRVMIICENPKHKQKQG

SEQ ID NO: 38 - protein, 37 aa, Unknown
  peptide for immunotherapy; SG-3-00802_variant 37 peptide
MKTRSSVKPMCDKCKVIKRKGRVAVICENPKHKQRQG

SEQ ID NO: 39 - protein, 37 aa, Unknown
  peptide for immunotherapy; SG-3-00802_variant 38 peptide
MKVRPSVKPMCDKCKVIKRKGKVMVICQEPKHKQRQG

SEQ ID NO: 40 - protein, 36 aa, Unknown
  peptide for immunotherapy; SG-3-00802_variant 39 peptide
KVRSSVKPICEKCKVIKRKGIVRVICENPKHKQRQG

SEQ ID NO: 41 - protein, 37 aa, Unknown
  peptide for immunotherapy; SG-3-00802_variant 40 peptide
MKVRASVKPICDKCKVIKRKGIVRVICENPKHKQRQG

SEQ ID NO: 42 - protein, 37 aa, Unknown
  peptide for immunotherapy; SG-3-00802_variant 41 peptide
MKVRPSVKKMCDKCKIIRRHGKILVICENPRHKQRQG

SEQ ID NO: 43 - protein, 37 aa, Unknown
  peptide for immunotherapy; SG-3-00802_variant 42 peptide
MKVRPSVKKMCDKCKVIKRKGKILVICENPKHKQRQG

SEQ ID NO: 44 - protein, 37 aa, Unknown
  peptide for immunotherapy; SG-3-00802_variant 43 peptide
MKVRSSVKPICEKCKVIKRKGSVRIICENPKHKQRQG

SEQ ID NO: 45 - protein, 38 aa, Artificial
  generic peptide for immunotherapy; generic SG-3-00802 peptide
  misc_feature (1)..(1): Xaa can be any naturally occurring amino acid
  misc_feature (3)..(5): Xaa can be any naturally occurring amino acid
  misc_feature (9)..(10): Xaa can be any naturally occurring amino acid
  misc_feature (12)..(13): Xaa can be any naturally occurring amino acid
  misc_feature (15)..(17): Xaa can be any naturally occurring amino acid
  misc_feature (19)..(19): Xaa can be any naturally occurring amino acid
  misc_feature (21)..(21): Xaa can be any naturally occurring amino acid
  misc_feature (23)..(26): Xaa can be any naturally occurring amino acid
  misc_feature (28)..(30): Xaa can be any naturally occurring amino acid
  misc_feature (32)..(32): Xaa can be any naturally occurring amino acid
  misc_feature (36)..(36): Xaa can be any naturally occurring amino acid
  misc_feature (38)..(38): Xaa can be any naturally occurring amino acid
XKXXXSVKXXCXXCXXXIXRXGXXXXIXXXPXHKQXQX

SEQ ID NO: 46 - protein, 37 aa, Artificial
  peptide for immunotherapy; SG-3-06683, retro-inverso peptide variant of SEQ ID NO: 1 optionally with some or all D-amino acids
GQRQKHKPNECIVMVRGKRRIIKCKECMPKVSPRVKM

SEQ ID NO: 47 - protein, 37 aa, Unknown
  peptide for immunotherapy; SG-3-01677
MKVRSSVKPICEKCKVIKRKGAIRVICENPKHKQRQG

SEQ ID NO: 48 - protein, 37 aa, Unknown
  peptide for immunotherapy; SG-3-01797
MKVRPSVKKMCDKCKIIRRHGKVLVICENPRHKQRQG

SEQ ID NO: 49 - protein, 37 aa, Unknown
  peptide for immunotherapy; SG-3-03562
MKVRPSVKPMCDKCRVIKRKGRVMIICENPKHKQRQG

SEQ ID NO: 50 - protein, 37 aa, Unknown; identical to SEQ ID NO: 3
  peptide for immunotherapy; SG-3-03785

SEQ ID NO: 51 - protein, 27 aa, Unknown
  peptide for immunotherapy; SG-3-05021
LGKTDWIEKYFKVKKEKIDKMQRFLQG

SEQ ID NO: 52 - skipped

SEQ ID NO: 53 - protein, 31 aa, Unknown
  peptide for immunotherapy; SG-3-04865
GDTIVEWYRTARARQTFLHKRTWQKNLRAFV

SEQ ID NO: 54 - protein, 20 aa, Unknown
  peptide for immunotherapy; SG-3-04534
MWSVVKKIIALSGDKNKYIK

SEQ ID NO: 55 - protein, 26 aa, Unknown
  peptide for immunotherapy; SG-3-04552
IRKKVDEKKKQLQKVQTSFHQKVSSI

SEQ ID NO: 56 - protein, 29 aa, Unknown
  peptide for immunotherapy; SG-3-04785
LYQQYIHPIVMSLLRKKSMTKPRVNTGVL

SEQ ID NO: 57 - protein, 19 aa, Unknown
  peptide for immunotherapy; SG-3-04520
MGPLIQWFQKNWIKKVIEK

SEQ ID NO: 58 - protein, 18 aa, Unknown
  peptide for immunotherapy; SG-3-04284
TKKIPWSFWKKSKNPAVR

SEQ ID NO: 59 - protein, 19 aa, Unknown
  peptide for immunotherapy; SG-3-04191
KNRIEKWWPCKKYLVAHRR

SEQ ID NO: 60 - protein, 18 aa, Unknown
  peptide for immunotherapy; SG-3-04414
MKEKIKKMPGRIRNYFCS

SEQ ID NO: 61 - protein, 18 aa, Unknown
  peptide for immunotherapy; SG-3-04260
NMSGWREFVKLVRKSPKS

SEQ ID NO: 62 - protein, 27 aa, Unknown
  peptide for immunotherapy; SG-3-04984
FLALLGGNGTGKTTTLKLLANLKKAYR

SEQ ID NO: 63 - protein, 18 aa, Unknown
  peptide for immunotherapy; SG-3-04487
MLKKILYYGKENNKKTVR

SEQ ID NO: 64 - protein, 37 aa, Unknown; identical to SEQ ID NO: 49
  peptide for immunotherapy; SG-3-03562

SEQ ID NO: 65 - protein, 27 aa, Unknown
  peptide for immunotherapy; SG-3-04392
MKNQSHPIVIVKKRKHKAHGHGSHGSW